(19) United States
(12) Patent Application Publication
(10) Pub. No.: US 2020/0121589 A1
(43) Pub. Date: Apr. 23, 2020

(54) COMPOSITIONS FOR DRUG DELIVERY AND METHODS OF USE THEREOF

(71) Applicant: Viramal Limited, London (GB)

(72) Inventors: William Bologna, New York, NY (US); Finn Larsen, Hawick (GB); Simona Fiore, London (GB)

(21) Appl. No.: 16/624,597

(22) PCT Filed: Jun. 22, 2018
(86) PCT No.: PCT/IB2018/000807

§ 371 Date: Dec. 19, 2019

Related U.S. Application Data
(60) Provisional application No. 62/523,510, filed on Jun. 22, 2017.

(51) Int. Cl. A61K 9/00; A61K 9/107; A61K 31/57; A61K 31/58; A61K 47/38; A61K 47/32; A61K 47/14; G01N 33/68; A61P 35/00; A61P 15/00; A61P 29/00; A61K 9/06; A61K 47/10; A61K 31/565

(52) U.S. Cl. CPC A61K 9/0034; A61K 9/107; A61K 31/57; A61K 31/58; A61K 47/38; A61K 47/32; A61K 31/565; G01N 33/68; A61P 35/00; A61P 15/00; A61P 29/00; A61K 9/06; A61K 47/10; A61K 47/14

(57) ABSTRACT

Disclosed herein are compositions for administering vaginally of an active agent or a salt to a subject comprising the active agent or salt thereof, a bioadhesive, and an emulsion. Also disclosed herein are methods treating a disease of condition by administering vaginally to a subject a pharmaceutical composition, and kits comprising the pharmaceutical composition.

### SUMMARY

Disclosed herein are methods comprising administering vaginally to a subject a pharmaceutical composition in the form of an emulsion that can comprise an active agent or salt thereof and a bioadhesive; where a pharmaceutical composition can be administered at a dose of from about 10 mg to about 400 mg of an active agent or salt thereof; and where an administering vaginally of a pharmaceutical composition produces a substantially zero order release rate profile of an active agent into a peritoneal cavity of a subject at least about 8 hours after an administering of a pharmaceutical composition. In some embodiments, a dose can comprise from about 50 mg to about 100 mg of an active agent or salt thereof. In some embodiments, an active agent or salt thereof can be present in a peritoneal cavity at about 12 hours after an administering of a pharmaceutical composition. In some embodiments, an administering vaginally of a pharmaceutical composition produces a peritoneal concentration of an active agent, a metabolite thereof, or a salt thereof that can be at least about 4-fold greater than a peritoneal concentration of an active agent, metabolite thereof, or salt thereof achieved through an oral administering of an oral pharmaceutical composition that can comprise a substantially equivalent dosage of an active agent or salt thereof. In some embodiments, the method can be a method of treating a disease or condition. In some embodiments, a disease or condition can be selected from the group consisting of an endometrial disorder, a cancer, an inflammatory disorder, an infection, and any combination thereof. In some embodiments, a disease or condition can be an endometrial disorder. In some embodiments, an endometrial disorder can be endometriosis, adenomyosis, or a combination thereof. In some embodiments, an amount of an endometrial deposit can be lower after an administering vaginally of a pharmaceutical composition than an amount prior to an administering vaginally of a pharmaceutical composition. In some embodiments, a disease or condition can be a cancer. In some embodiments, a cancer can be selected from the group consisting of cervical cancer; ovarian cancer; mesothelial cancer; peritoneal cancer; and any combination thereof. In some embodiments, a treating can comprise a reduction of a tumor size or a reduction of a tumor growth. In some embodiments, a reduction of a tumor size or a reduction of a tumor growth can be determined by a reduction in a tumor volume as measured by ultrasound. In some embodiments, a disease or condition can be an inflammatory disorder. In some embodiments, an inflammatory disorder can be selected from the group consisting of: pelvic inflammatory/infectious disease; chronic pelvic pain; and any combination thereof. In some embodiments, a treating can comprise reducing an amount of at least one pro-inflammatory cytokine to an amount that can be lower than prior to an administering vaginally of a pharmaceutical composition. In some embodiments, a disease or condition can be an infection. In some embodiments, an infection can be a bacterial infection. In some embodiments, an infection can be a viral infection. In some embodiments, an infection can be a fungal infection. In some embodiments, an active agent or salt thereof can be selected from the group consisting of a hormone; an antineoplastic; a GnRH agonist; a GnRH antagonist; a steroid; an analgesic; an antibiotic; an antiviral compound; an antifungal compound; an anti-inflammatory; a salt of any of these; and any combination thereof. In some embodiments, an active agent can be a hormone or a salt thereof. In some embodiments, a hormone or salt thereof can be selected from the group consisting of: estradiol; ethinylestradiol; progesterone; levonorgestrel; desogestrel; a synthetic progesterone; a salt of any of these; and any combination thereof. In some embodiments, an active agent can be an antineoplastic or a salt thereof. In some embodiments, an antineoplastic or salt thereof can be selected from the group consisting of cyclophosphamide; methotrexate; 5-fluorouracil; doxorubicin; procarbazine; prednisolone; bleomycin; vinblastine; dacarbazine; cisplatin; epirubicin; dichloroacetate a salt of any of these; and any combination thereof. In some embodiments, an active agent can be a GnRH agonist or a GnRH antagonist or a salt thereof. In some embodiments, a GnRH agonist or a GnRH antagonist or salt thereof can be selected from the group consisting of leuprolide; buserelin; histrelin; goserelin; deslorelin; nafarelin; triptorelin; cetrorelix, abarelix; ganirelix, ozarelix, degarelix or teverelix or a salt of any of these; and any combination thereof. In some embodiments, an active agent can be a steroid or a salt thereof. In some embodiments, a steroid or salt thereof can be danazol or a salt thereof. In some embodiments, an active agent can be an antibiotic or a salt thereof. In some embodiments, an antibiotic or salt thereof can be selected from the group consisting of Ceftobiprole; Ceftaroline; Clindamycin; Dalbavancin; Daptomycin; Linezolid; Mupirocin; Oritavancin; Tedizolid; Telavancin; Tigecycline; Vancomycin; an Aminoglycoside; a Carbapenem; Ceftazidime; Cefepime; Ceftobiprole; a Fluoroquinolone; Piperacillin; Ticarcillin; Linezolid; a Streptogramin; Tigecycline; Daptomycin; a salt of any of these; and any combination thereof. In some embodiments, an active agent can be an antiviral compound or a salt thereof. In some embodiments, an antiviral compound or salt thereof can be selected from the group consisting of Ceftobiprole; Ceftaroline; Clindamycin; Dalbavancin; Daptomycin; Linezolid; Mupirocin; Oritavancin; Tedizolid; Telavancin; Tigecycline; Vancomycin; an Aminoglycoside; a Carbapenem; Ceftazidime; Cefepime; Ceftobiprole; a Fluoroquinolone; Piperacillin; Ticarcillin; Linezolid; a Streptogramin; Tigecycline; Daptomycin; a salt of any of these; and any combination thereof. In some embodiments, an active agent can be an antifungal compound or a salt thereof. In some embodiments, an antifungal compound or salt thereof can be selected from the group consisting of ciclopirox olamine; haloprogin; tolnaftate; undecylenate; topical nysatin; amorolfine; butenafine; naftifine; terbinafine; a salt of any of these; and any combination thereof. In some embodiments, an active agent can be an anti-inflammatory or a salt thereof. In some embodiments, an anti-inflammatory or salt thereof can be selected from the group consisting of diclofenac; ketoprofen; ibuprofen; aspirin; a salt of any of these; and any combination thereof. In some embodiments, a pharmaceutical composition can be administered at a dose of an active agent or salt thereof of at least at least about 50 mg, at least about 100 mg, at least about 150 mg, at least about 200 mg, at least about 250 mg, at least about 300 mg, at least about 350 mg, or at least about 400 mg. In some embodiments, a pharmaceutical composition can be administered at a dose of an active agent or salt thereof per body weight of a subject o at least about 0.1 mg/kg, at least about 0.5 mg/kg, at least about 1 mg/kg, at least about 1.5 mg/kg, at least about 2 mg/kg, at least about 2.5 mg/kg, at least about 3 mg/kg, at least about 3.5 mg/kg, at least about 4 mg/kg, at least about 4.5 mg/kg, at least about 5 mg/kg, at least about 5.5 mg/kg, at least about 6 mg/kg, at least about 6.5 mg/kg, at least about 7 mg/kg, at least about 7.5 mg/kg, at least about 8 mg/kg, at least about 8.5 mg/kg, at least about 9 mg/kg, at least about 9.5 mg/kg, at least about 10 mg/kg, at least about 11 mg/kg, at least about 12 mg/kg, at least about 13 mg/kg, at least about 14 mg/kg, at least about 15 mg/kg, at least about 16 mg/kg, at least about 17 mg/kg, at least about 18 mg/kg, at least about 19 mg/kg, or at least about 20 mg/kg. In some embodiments, a pharmaceutical composition can comprise a substantially uniform mixture of an organic phase and an aqueous phase. In some embodiments, an organic phase can comprise at least one oleogel that can comprise at least one oily agent and at least one water insoluble cellulose polymer. In some embodiments, a water insoluble cellulose polymer can be an alkyl cellulose. In some embodiments, an alkyl cellulose can be selected from the group consisting of methylcellulose; ethylcellulose; hydroxypropylcellulose; and any combination thereof. In some embodiments, a water insoluble cellulose polymer can be an alkyl carboxylic containing cellulose or a salt thereof. In some embodiments, an alkyl carboxylic acid containing cellulose can be a substantially non-sodium containing carboxymethylcellulose. In some embodiments, a substantially non-sodium containing carboxymethylcellulose can comprise from about 1% to about 10% by weight of a total weight of a pharmaceutical composition. In some embodiments, an alkyl cellulose can comprise from about 1% to about 10% by weight of a total weight of a pharmaceutical composition. In some embodiments, an ethylcellulose can comprise from about 1% to about 10% by weight of a total weight of a pharmaceutical composition. In some embodiments, an alkyl carboxylic containing cellulose or a salt thereof can comprise from about 1% to about 10% by weight of a total weight of a pharmaceutical composition. In some embodiments, an aqueous phase can comprise at least one aqueous gel. In some embodiments, an aqueous gel can further comprise at least one gelling agent. In some embodiments, the at least one gelling agent can be selected from the group consisting of a carbomer; a poloxamer; sodium carboxymethylcellulose; and a combination thereof. In some embodiments, the at least one gelling agent can comprise from about 0.1% to about 10% by weight of a total weight of an aqueous gel. In some embodiments, the at least one gelling agent can comprise from about 0.01% to about 10% by weight of a total weight of a pharmaceutical composition. In some embodiments, an oily agent can be selected from the group consisting of: a monoglyceride; a diglyceride; a triglyceride; and any combination thereof. In some embodiments, an oily agent can be isolated and purified. In some embodiments, an oily agent can be selected from the group consisting of: a synthetic diglyceride; a synthetic triglyceride; a propylene glycol isostearate; a polyoxyethylenated oleic glyceride mixture; an oil of plant origin; and any combination thereof. In some embodiments, a propylene glycol isostearate can comprise from about 0.2% to about 2% by weight of a total weight of a pharmaceutical composition. In some embodiments, a polyoxyethylenated oleic glyceride mixture can comprise from about 0.2% to about 2% by weight of a total weight of a pharmaceutical composition. In some embodiments, an organic phase can be at a ratio of from about 10:90 to about 90:10 by weight with respect to an aqueous phase. In some embodiments, a bioadhesive can be selected from the group consisting of: a carbomer; glyceryl monooleate; hypromellose; polycarbophil; poly(methylvinyl ether-co-maleic anhydride); a salt thereof; and a combination thereof. In some embodiments, a bioadhesive can be polycarbophil, a salt thereof, or a combination thereof. In some embodiments, a pharmaceutical composition can comprise a concentration of an alcohol from about 0% to about 4% by weight based on a total weight of a pharmaceutical composition, where an alcohol can be ethanol or isopropanol. In some embodiments, a concentration of alcohol can be about 3.5% by weight based on a total weight of a pharmaceutical composition. In some embodiments, an active agent can comprise from about 0.00001% to about 10% by weight of a total weight of a pharmaceutical composition. In some embodiments, a pharmaceutical composition can comprise from about 0% to about 4% of a penetration enhancer by weight of a total weight of a pharmaceutical composition. In some embodiments, a pharmaceutical composition can comprise from about 0% to about 2% of a surfactant by weight of a total weight of a pharmaceutical composition, where a surfactant can be selected from the group consisting of non-ionic; cationic; amphoteric; zwitterionic; and any combination thereof. In some embodiments, a pharmaceutical composition can be administered to a subject in unit dose form. In some embodiments, an administering vaginally of a pharmaceutical composition can be performed about every hour, about every 4 hours, about every 8 hours, about every 12 hours, or about every 24 hours. In some embodiments, an administering vaginally of a pharmaceutical composition can be performed about once, about twice, about 3 times, about 4 times, about 5 times, about 6 times, about 7 times, or about 8 times within 24 hours. In some embodiments, an administering vaginally of a pharmaceutical composition can be performed about once, about twice, about 3 times, about 4 times, about 5 times, about 6 times, about 7 times, about 8 times, about 9 times, about 10 times, about 11 times, about 12 times, about 13 times, about 14 times, about 15 times, about 16 times, about 17 times, about 18 times, about 19 times, or about 20 times a week. In some embodiments, an administering vaginally of a pharmaceutical composition can be performed about once, about twice, about 3 times, about 4 times, about 5 times, about 6 times, about 7 times, about 8 times, about 9 times, about 10 times, about 11 times, about 12 times, about 13 times, about 14 times, about 15 times, about 16 times, about 17 times, about 18 times, about 19 times, about 20 times, about 21 times, about 22 times, about 23 times, about 24 times, about 25 times, about 26 times, about 27 times, about 28 times, about 29 times, about 30 times, or about 31 times a month. In some embodiments, a pharmaceutical composition can be applied with a finger. In some embodiments, a pharmaceutical composition can be applied with a glove. In some embodiments, a pharmaceutical composition can be applied with an applicator. In some embodiments, an administering vaginally can comprise an administering intravaginally, an administering topically, an administering by suppository, or any combination thereof. In some embodiments, a pharmaceutical composition maintains a substantially stable uniform appearance over a period of about 1 year when stored in a sealed container, at about 25° C., at about 1 atm pressure, and at about 50% relative humidity. In some embodiments, an administering vaginally of a pharmaceutical composition produces a greater ability to achieve a pregnancy after about 6 months from a terminal administering vaginally of a pharmaceutical composition; relative to an administering orally of an oral pharmaceutical composition that can comprise a substantially equivalent amount of an active agent or salt thereof.

Also disclosed herein are methods comprising administering vaginally to a subject a pharmaceutical composition in the form of an emulsion that can comprise: an active agent or salt thereof and a bioadhesive; where an administering vaginally of a pharmaceutical composition can comprise an administering of a dose of an active agent or salt thereof; and where an administering vaginally at least partially minimizes a side effect relative to an administering orally of an oral pharmaceutical composition that can comprise a substantially equivalent dose of an active agent or salt thereof. In some embodiments, an administering vaginally can be performed at least 2 times within 24 hours. In some embodiments, a side effect can be selected from the group consisting of cardiotoxicity; renal toxicity; hepatotoxicity; and any combination thereof as determined by a lower amount of a biomarker implicated in a side effect after an administering vaginally of a pharmaceutical composition relative to an amount of a biomarker implicated in a side effect after an administering orally of an oral pharmaceutical composition. In some embodiments, an administering vaginally of a pharmaceutical composition produces a peritoneal concentration of an active agent, a metabolite thereof, or a salt thereof that can be at least about 4-fold greater than a peritoneal concentration of an active agent, metabolite thereof, or salt thereof achieved through an oral administering of an oral pharmaceutical composition that can comprise a substantially equivalent dosage of an active agent or salt thereof. In some embodiments, the method can be a method of treating a disease or condition. In some embodiments, a disease or condition can be selected from the group consisting of an endometrial disorder, a cancer, an inflammatory disorder, an infection, and any combination thereof. In some embodiments, a disease or condition can be an endometrial disorder. In some embodiments, an endometrial disorder can be endometriosis, adenomyosis, or a combination thereof. In some embodiments, an amount of an endometrial deposit can be lower after an administering vaginally of a pharmaceutical composition than an amount prior to an administering vaginally of a pharmaceutical composition. In some embodiments, a disease or condition can be a cancer. In some embodiments, a cancer can be selected from the group consisting of cervical cancer; ovarian cancer; mesothelial cancer; peritoneal cancer; and any combination thereof. In some embodiments, a treating can comprise a reduction of a tumor size or a reduction of a tumor growth. In some embodiments, a reduction of a tumor size or a reduction of a tumor growth can be determined by a reduction in a tumor volume as measured by ultrasound. In some embodiments, a disease or condition can be an inflammatory disorder. In some embodiments, an inflammatory disorder can be selected from the group consisting of: pelvic inflammatory disease; chronic pelvic pain; and any combination thereof. In some embodiments, a treating can comprise reducing an amount of at least one pro-inflammatory cytokine to an amount that can be lower than prior to an administering vaginally of a pharmaceutical composition. In some embodiments, a disease or condition can be an infection. In some embodiments, an infection can be a bacterial infection. In some embodiments, an infection can be a viral infection. In some embodiments, an infection can be a fungal infection. In some embodiments, an active agent or salt thereof can be selected from the group consisting of a hormone; an antineoplastic; a GnRH agonist; a GnRH antagonist; a steroid; an analgesic; an antibiotic; an antiviral compound; an antifungal compound; an anti-inflammatory; a salt of any of these; and any combination thereof. In some embodiments, an active agent can be a hormone or a salt thereof. In some embodiments, a hormone or salt thereof can be selected from the group consisting of: estradiol; ethinylestradiol; progesterone; levonorgestrel; desogestrel; a synthetic progesterone; a salt of any of these; and any combination thereof. In some embodiments, an active agent can be an antineoplastic or a salt thereof. In some embodiments, an antineoplastic or salt thereof can be selected from the group consisting of cyclophosphamide; methotrexate; 5-fluorouracil; doxorubicin; procarbazine; prednisolone; bleomycin; vinblastine; dacarbazine; cisplatin; epirubicin; dichloroacetate; a salt of any of these; and any combination thereof. In some embodiments, an active agent can be a GnRH agonist, a GnRH antagonist, or a salt thereof. In some embodiments, a GnRH agonist or salt thereof can be selected from the group consisting of leuprolide; buserelin; histrelin; goserelin; deslorelin; nafarelin; triptorelin; a salt of any of these; and any combination thereof. In some embodiments, an active agent can be a steroid or a salt thereof. In some embodiments, a steroid or salt thereof can be danazol or a salt thereof. In some embodiments, a subject can be monitored for a period of at least 12 months after a termination of an administering. In some embodiments, an active agent can be an antibiotic or a salt thereof. In some embodiments, an antibiotic or salt thereof can be selected from the group consisting of Ceftobiprole; Ceftaroline; Clindamycin; Dalbavancin; Daptomycin; Linezolid; Mupirocin; Oritavancin; Tedizolid; Telavancin; Tigecycline; Vancomycin; an Aminoglycoside; a Carbapenem; Ceftazidime; Cefepime; Ceftobiprole; a Fluoroquinolone; Piperacillin; Ticarcillin; Linezolid; a Streptogramin; Tigecycline; Daptomycin; a salt of any of these; and any combination thereof. In some embodiments, an active agent can be an antiviral compound or a salt thereof. In some embodiments, an antiviral compound or salt thereof can be selected from the group consisting of Ceftobiprole; Ceftaroline; Clindamycin; Dalbavancin; Daptomycin; Linezolid; Mupirocin; Oritavancin; Tedizolid; Telavancin; Tigecycline; Vancomycin; an Aminoglycoside; a Carbapenem; Ceftazidime; Cefepime; Ceftobiprole; a Fluoroquinolone; Piperacillin; Ticarcillin; Linezolid; a Streptogramin; Tigecycline; Daptomycin; a salt of any of these; and any combination thereof. In some embodiments, an active agent can be an antifungal compound or a salt thereof. In some embodiments, an antifungal compound or salt thereof can be selected from the group consisting of ciclopirox olamine; haloprogin; tolnaftate; undecylenate; topical nysatin; amorolfine; butenafine; naftifine; terbinafine; a salt of any of these; and any combination thereof. In some embodiments, an active agent can be an anti-inflammatory or a salt thereof. In some embodiments, an anti-inflammatory or salt thereof can be selected from the group consisting of diclofenac; ketoprofen; ibuprofen; aspirin; a salt of any of these; and any combination thereof. In some embodiments, a pharmaceutical composition can be administered at a dose of an active agent or salt thereof of at least at least about 50 mg, at least about 100 mg, at least about 150 mg, at least about 200 mg, at least about 250 mg, at least about 300 mg, at least about 350 mg, or at least about 400 mg. In some embodiments, a pharmaceutical composition can be administered at a dose of an active agent or salt thereof per body weight of a subject of at least about 0.1 mg/kg, at least about 0.5 mg/kg, at least about 1 mg/kg, at least about 1.5 mg/kg, at least about 2 mg/kg, at least about 2.5 mg/kg, at least about 3 mg/kg, at least about 3.5 mg/kg, at least about 4 mg/kg, at least about 4.5 mg/kg, at least about 5 mg/kg, at least about 5.5 mg/kg, at least about 6 mg/kg, at least about 6.5 mg/kg, at least about 7 mg/kg, at least about 7.5 mg/kg, at least about 8 mg/kg, at least about 8.5 mg/kg, at least about 9 mg/kg, at least about 9.5 mg/kg, at least about 10 mg/kg, at least about 11 mg/kg, at least about 12 mg/kg, at least about 13 mg/kg, at least about 14 mg/kg, at least about 15 mg/kg, at least about 16 mg/kg, at least about 17 mg/kg, at least about 18 mg/kg, at least about 19 mg/kg, or at least about 20 mg/kg. In some embodiments, a pharmaceutical composition can comprise a substantially uniform mixture of an organic phase and an aqueous phase. In some embodiments, an organic phase can comprise at least one oleogel that can comprise at least one oily agent and at least one water insoluble cellulose polymer. In some embodiments, a water insoluble cellulose polymer can be an alkyl cellulose. In some embodiments, an alkyl cellulose can be selected from the group consisting of methylcellulose; ethylcellulose; hydroxypropylcellulose; and any combination thereof. In some embodiments, a water insoluble cellulose polymer can be an alkyl carboxylic containing cellulose or a salt thereof. In some embodiments, an alkyl carboxylic acid containing cellulose can be a substantially non-sodium containing carboxymethylcellulose. In some embodiments, a substantially non-sodium containing carboxymethylcellulose can comprise from about 1% to about 10% by weight of a total weight of a pharmaceutical composition. In some embodiments, an alkyl cellulose can comprise from about 1% to about 10% by weight of a total weight of a pharmaceutical composition. In some embodiments, an ethylcellulose can comprise from about 1% to about 10% by weight of a total weight of a pharmaceutical composition. In some embodiments, an alkyl carboxylic containing cellulose or a salt thereof can comprise from about 1% to about 10% by weight of a total weight of a pharmaceutical composition. In some embodiments, an aqueous phase can comprise at least one aqueous gel. In some embodiments, an aqueous gel can further comprise at least one gelling agent. In some embodiments, the at least one gelling agent can be selected from the group consisting of a carbomer; a poloxamer; sodium carboxymethylcellulose; and a combination thereof. In some embodiments, the at least one gelling agent can comprise from about 0.1% to about 10% by weight of a total weight of an aqueous gel. In some embodiments, the at least one gelling agent can comprise from about 0.01% to about 10% by weight of a total weight of a pharmaceutical composition. In some embodiments, an oily agent can be selected from the group consisting of: a monoglyceride; a diglyceride; a triglyceride; and any combination thereof. In some embodiments, an oily agent can be isolated and purified. In some embodiments, an oily agent can be selected from the group consisting of: a synthetic diglyceride; a synthetic triglyceride; a propylene glycol isostearate; a polyoxyethylenated oleic glyceride mixture; an oil of plant origin; and any combination thereof. In some embodiments, a propylene glycol isostearate can comprise from about 0.2% to about 2% by weight of a total weight of a pharmaceutical composition. In some embodiments, a polyoxyethylenated oleic glyceride mixture can comprise from about 0.2% to about 2% by weight of a total weight of a pharmaceutical composition. In some embodiments, an organic phase can be at a ratio of from about 10:90 to about 90:10 by weight with respect to an aqueous phase. In some embodiments, a bioadhesive can be selected from the group consisting of: a carbomer; glyceryl monooleate; hypromellose; polycarbophil; poly(methylvinyl ether-co-maleic anhydride); a salt thereof; and a combination thereof. In some embodiments, a bioadhesive can be polycarbophil, a salt thereof, or a combination thereof. In some embodiments, a pharmaceutical composition can comprise a concentration of an alcohol from about 0% to about 4% by weight based on a total weight of a pharmaceutical composition, where an alcohol can be ethanol or isopropanol. In some embodiments, a concentration of alcohol can be about 3.5% by weight based on a total weight of a pharmaceutical composition. In some embodiments, an active agent can comprise from about 0.00001% to about 10% by weight of a total weight of a pharmaceutical composition. In some embodiments, a pharmaceutical composition can comprise from about 0% to about 4% of a penetration enhancer by weight of a total weight of a pharmaceutical composition. In some embodiments, a pharmaceutical composition can comprise from about 0% to about 2% of a surfactant by weight of a total weight of a pharmaceutical composition, where a surfactant can be selected from the group consisting of non-ionic; cationic; amphoteric; zwitterionic; and any combination thereof. In some embodiments, a pharmaceutical composition can be administered to a subject in unit dose form. In some embodiments, an administering vaginally of a pharmaceutical composition can be performed about every hour, about every 4 hours, about every 8 hours, about every 12 hours, or about every 24 hours. In some embodiments, an administering vaginally of a pharmaceutical composition can be performed about once, about twice, about 3 times, about 4 times, about 5 times, about 6 times, about 7 times, or about 8 times within 24 hours. In some embodiments, an administering vaginally of a pharmaceutical composition can be performed about once, about twice, about 3 times, about 4 times, about 5 times, about 6 times, about 7 times, about 8 times, about 9 times, about 10 times, about 11 times, about 12 times, about 13 times, about 14 times, about 15 times, about 16 times, about 17 times, about 18 times, about 19 times, or about 20 times a week. In some embodiments, an administering vaginally of a pharmaceutical composition can be performed about once, about twice, about 3 times, about 4 times, about 5 times, about 6 times, about 7 times, about 8 times, about 9 times, about 10 times, about 11 times, about 12 times, about 13 times, about 14 times, about 15 times, about 16 times, about 17 times, about 18 times, about 19 times, about 20 times, about 21 times, about 22 times, about 23 times, about 24 times, about 25 times, about 26 times, about 27 times, about 28 times, about 29 times, about 30 times, or about 31 times a month. In some embodiments, a pharmaceutical composition can be applied with a finger. In some embodiments, a pharmaceutical composition can be applied with a glove. In some embodiments, a pharmaceutical composition can be applied with an applicator. In some embodiments, an administering vaginally can comprise an administering intravaginally, an administering topically, an administering by suppository, or any combination thereof. In some embodiments, a pharmaceutical composition maintains a substantially stable uniform appearance over a period of about 1 year when stored in a sealed container, at about 25° C., at about 1 atm pressure, and at about 50% relative humidity. In some embodiments, an administering vaginally of a pharmaceutical composition produces a greater ability to achieve a pregnancy after about 6 months from a terminal administering vaginally of a pharmaceutical composition; relative to an administering orally of an oral pharmaceutical composition that can comprise a substantially equivalent amount of an active agent or salt thereof.

Also disclosed herein are pharmaceutical compositions comprising: (a) at least one oleogel that can comprise at least one oily agent and at least one water insoluble cellulose polymer; (b) at least one aqueous gel; (c) an active agent or salt thereof and (d) a bioadhesive; where an active agent or salt thereof can be present in a pharmaceutical composition in an amount of from about 50 mg to about 400 mg. In some embodiments, the at least one oleogel and the at least one aqueous gel can be in the form of an emulsion. In some embodiments, an active agent or salt thereof can be selected from the group consisting of a hormone; an antineoplastic; a GnRH agonist; a GnRH antagonist; a steroid; an analgesic; an antibiotic; an antiviral compound; an antifungal compound; an anti-inflammatory; a salt of any of these; and any combination thereof. In some embodiments, an active agent can be a hormone or a salt thereof. In some embodiments, a hormone or salt thereof can be selected from the group consisting of: testosterone; estradiol; ethinylestradiol; progesterone; levonorgestrel; desogestrel; a synthetic progesterone; a salt of any of these; and any combination thereof. In some embodiments, an active agent can be an antineoplastic or a salt thereof. In some embodiments, an antineoplastic or salt thereof can be selected from the group consisting of cyclophosphamide; methotrexate; 5-fluorouracil; doxorubicin; procarbazine; prednisolone; bleomycin; vinblastine; dacarbazine; cisplatin; epirubicin; dichloroacetate; a salt of any of these; and any combination thereof. In some embodiments, an active agent can be a GnRH agonist, a GnRH antagonist, or a salt thereof. In some embodiments, a GnRH agonist, a GnRH antagonist or salt thereof can be selected from the group consisting of leuprolide; buserelin; histrelin; goserelin; deslorelin; nafarelin; triptorelin; cetrorelix, abarelix; ganirelix, ozarelix, degarelix or teverelix; a salt of any of these; and any combination thereof. In some embodiments, an active agent can be a steroid or a salt thereof. In some embodiments, a steroid or salt thereof can be danazol or a salt thereof. In some embodiments, an active agent can be an antibiotic or a salt thereof. In some embodiments, an antibiotic or salt thereof can be selected from the group consisting of Ceftobiprole; Ceftaroline; Clindamycin; Dalbavancin; Daptomycin; Linezolid; Mupirocin; Oritavancin; Tedizolid; Telavancin; Tigecycline; Vancomycin; an Aminoglycoside; a Carbapenem; Ceftazidime; Cefepime; Ceftobiprole; a Fluoroquinolone; Piperacillin; Ticarcillin; Linezolid; a Streptogramin; Tigecycline; Daptomycin; a salt of any of these; and any combination thereof. In some embodiments, an active agent can be an antiviral compound or a salt thereof. In some embodiments, an antiviral compound or salt thereof can be selected from the group consisting of Ceftobiprole; Ceftaroline; Clindamycin; Dalbavancin; Daptomycin; Linezolid; Mupirocin; Oritavancin; Tedizolid; Telavancin; Tigecycline; Vancomycin; an Aminoglycoside; a Carbapenem; Ceftazidime; Cefepime; Ceftobiprole; a Fluoroquinolone; Piperacillin; Ticarcillin; Linezolid; a Streptogramin; Tigecycline; Daptomycin; a salt of any of these; and any combination thereof. In some embodiments, an active agent can be an antifungal compound or a salt thereof. In some embodiments, an antifungal compound or salt thereof can be selected from the group consisting of ciclopirox olamine; haloprogin; tolnaftate; undecylenate; topical nysatin; amorolfine; butenafine; naftifine; terbinafine; a salt of any of these; and any combination thereof. In some embodiments, an active agent can be an anti-inflammatory or a salt thereof. In some embodiments, an anti-inflammatory or salt thereof can be selected from the group consisting of diclofenac; ketoprofen; ibuprofen; aspirin; a salt of any of these; and any combination thereof.

Also disclosed herein are kits comprising a container that can comprise a pharmaceutical described herein and instructions for use.

Also disclosed herein are methods of making a kit comprising placing a pharmaceutical composition described herein in a container.

Also disclosed herein are methods of detecting danazol or a salt thereof in a sample comprising: (a) contacting a portion of a sample from a subject with an albumin; and (b) detecting danazol or a salt thereof by mass spectrometry. In some embodiments, a detecting can comprise a determination of an amount of an [M+H]⁺ ion that can comprise an m/z of 338. In some embodiments, a detecting can comprise a comparison of an amount of an [M+H]⁺ ion that can comprise a m/z of 338 to an amount of an [M+H]⁺ ion from an internal standard; where an internal standard can be 19-Norethindrone or a salt thereof. In some embodiments, an albumin can be human serum albumin. In some embodiments, a sample can be dried and reconstituted in a buffer between (a) and (b).

Also disclosed herein are kits for determining an amount of danazol in a sample comprising: (a) a sample collection vessel; (b) an albumin; and (c) instructions for use. In some embodiments, a kit can further comprise an internal standard; where an internal standard can be 19-Norethindrone or a salt thereof. In some embodiments, a kit can further comprise a buffer. In some embodiments, instructions for use direct a user to: (a) collect a sample in a sample collection vessel; (b) contact a portion of a sample with an amount of an albumin; and (c) detect danazol or a salt thereof by mass spectrometry.

### BRIEF DESCRIPTION OF THE DRAWINGS

The novel features are set forth with particularity in the appended claims. A better understanding of features and advantages will be obtained by reference to the following detailed description that sets forth illustrative embodiments, in which exemplary principles are utilized, and the accompanying drawings of which:

FIG. 1 depicts an illustration of the peritoneal cavity and organs contained therein.

### DETAILED DESCRIPTION

### Overview

Disclosed herein are compositions and methods for delivering drug vaginally, in order to reach the pelvic area in such a way that high peritoneal fluid and tissue concentrations can be achieved with low detectable levels in the systemic circulation. In some cases, concentrations of at least 2 ng/mL, at least 5 ng/mL; at least >10 ng/mL, or even greater can be achieved. Comparing to other delivery method (e.g., oral administration), the drug delivery method disclosed herein can improve therapeutic efficacy. In some cases, such a delivery can maintain a substantially zero-order release profile of a therapeutically effective amount of an active agent or salt thereof into a peritoneal fluid for a given time interval when applied vaginally to a subject.

Also disclosed herein are methods of achieving a desired pharmacokinetic profile upon vaginally delivering to a subject a pharmaceutical composition. In some cases, the pharmaceutical composition can comprise an emulsion that can be admixed with an active agent and a bioadhesive.

An administering vaginally of a pharmaceutical composition described herein can be used to locally deliver an active agent or a salt thereof to a peritoneal cavity. Such a deliver can be used in lieu of systemic delivery, thereby decreasing an amount of the active agent or salt thereof present in circulation after administering vaginally of the pharmaceutical composition, relative to a systemic administering (such as orally administering) of a pharmaceutical composition that can comprise a substantially equivalent dose of the active agent or salt thereof. In some instances, the method described herein can reduce a potential for systemic adverse events and unwanted side effects that can occur with systemic administering of an active agent or salt thereof. Detection of an amount of an active agent or salt thereof in a subject sample is also contemplated using an assay employing detection of an albumin conjugate using mass spectrometry.

Administering vaginally of a pharmaceutical composition can be used to treat a disease or a condition in a subject. In some cases, a subject can be a subject in need of such a treatment, such as a subject who may be suspected of harboring, or has been previously been diagnosed with, a disease or condition treatable by administering vaginally to the subject a pharmaceutical composition described herein. A pharmaceutical composition described herein can include at least one active agent that can be selected based on the disease or condition to be treated.

Also disclosed herein are kits that can comprise a pharmaceutical composition described herein. Such a kit can include instructions for application of a pharmaceutical composition, and means for applying the pharmaceutical composition.

### Definitions

The terminology used herein is for the purpose of describing particular cases only and is not intended to be limiting. As used herein, the singular forms “a”, “an” and “the” can be intended to include the plural forms as well, unless the context clearly indicates otherwise. Furthermore, to the extent that the terms “including”, “includes”, “having”, “has”, “with”, or variants thereof can be used in either the detailed description and/or the claims, such terms can be intended to be inclusive in a manner similar to the term “comprising”.

The term “about” or “approximately” can mean within an acceptable error range for the particular value as determined by one of ordinary skill in the art, which will depend in part on how the value is measured or determined, i.e., the limitations of the measurement system. For example, “about” can mean about plus or minus 10%, per the practice in the art. Alternatively, “about” can mean a range of up to 20%, up to 10%, up to 5%, or up to 1% of a given value. Alternatively, particularly with respect to biological systems or processes, the term can mean within an order of magnitude, within 5-fold, or within 2-fold, of a value. Where particular values may be described in the application and claims, unless otherwise stated the term “about” meaning within an acceptable error range for the particular value should be assumed. Also, where ranges and/or subranges of values are provided, the ranges and/or subranges can include the endpoints of the ranges and/or subranges.

The term “substantially” as used herein can refer to a value approaching 100% of a given value. For example, an active agent that is “substantially localized” in an organ can indicate that about 90% by weight of an active agent, salt, or metabolite can be present in an organ relative to a total amount of an active agent, salt, or metabolite. In some cases, the term can refer to an amount that can be at least about 90%, 91%, 92%, 93%, 94%, 95%, 96%, 97%, 98%, 99%, 99.9%, or 99.99% of a total amount. In some cases, the term can refer to an amount that can be about 100% of a total amount.

The term “subject”, “patient” or “individual” as used herein can encompass a mammal and a non-mammal. A mammal can be any member of the Mammalian class, including but not limited to a human, a non-human primates such as a chimpanzee, an ape or other monkey species; a farm animal such as cattle, a horse, a sheep, a goat, a swine; a domestic animal such as a rabbit, a dog (or a canine), and a cat (or a feline); a laboratory animal including a rodent, such as a rat, a mouse and a guinea pig, and the like. A non-mammal can include a bird, a fish and the like. In some embodiments, a subject can be a mammal. In some embodiments, a subject can be a human. In some instances, a human can be an adult. In some instances, a human can be a child. In some instances, a human can be age 0-18 years old. In some instances, a human can be age 18-130 years. In some instances, a subject can be a female. In some instances, a subject can be diagnosed with, or can be suspected of having, a condition or disease. A subject can be a patient. A subject can be an individual. In some instances, a subject, patient or individual can be used interchangeably.

The term “preventing” can mean preventing additional symptoms, ameliorating or preventing the underlying metabolic causes of symptoms, and can include prophylaxis.

In some instances, “treat,” “treating”, “treatment,” “ameliorate” or “ameliorating” and other grammatical equivalents can include prophylaxis. “Treat,” “treating”, “treatment,” “ameliorate” or “ameliorating” and other grammatical equivalents can further include achieving a therapeutic benefit and/or a prophylactic benefit. Therapeutic benefit can mean eradication of the underlying disease being treated. Also, a therapeutic benefit can be achieved with the eradication of one or more of the physiological symptoms associated with the underlying disease such that an improvement can be observed in a subject notwithstanding that, in some embodiments, the subject can still be afflicted with the underlying disease.

The terms “effective amount”, “therapeutically effective amount” or “pharmaceutically effective amount” as used herein, can refer to a sufficient amount of a compound being administered which will at least partially ameliorate a symptom of a disease or condition being treated.

The terms “compound”, “agent”, “active agent”, or active ingredient can be used to refer to a drug or therapeutic as described herein. In some cases, the terms “additional compound”, “additional agent”, or “additional therapeutic agent” can be used interchangeably to refer to other active compounds, agents, or therapeutics that may be used in a composition described herein.

The terms “administer,” “administering”, “administration,” and the like, as used herein, can refer to methods that can be used to enable delivery of compounds or compositions to the desired site of biological action. These methods can include oral administration, intraduodenal administration, parenteral administration (including intravenous, subcutaneous, intrathecal, intraperitoneal, intramuscular, intravascular or infusion), topical and rectal administration. In some instances, a subject can administer the gel composition in the absence of supervision. In some instances, a subject can administer the gel composition under the supervision of a medical professional (e.g., a physician, nurse, physician's assistant, orderly, hospice worker, etc.).

In some exemplary embodiments, the administering can be a vaginal administering. A vaginal administering can include administering to any surface of a vagina. In some cases, a vaginal administering can be an intravaginal administering. An administering vaginally can include applying a composition described herein to a vagina using a finger or an applicator. An administering vaginally can include an administering intravaginally, an administering topically to a vagina, and a combination of administrations. Administering vaginally can also include administering via a carrier such as a patch, a suppository, an implantable depot, a tablet and the like.

The terms “pharmaceutically acceptable salt” or simply “salt” as used herein, can refer to a salt that retains at least some of the biological effectiveness of the free acids and bases of the specified compound. In some instances, the salt can be not biologically or otherwise undesirable. In some embodiments, a compound disclosed herein can possess acidic or basic groups and therefore can react with any of a number of inorganic or organic bases, and inorganic and organic acids, to form a pharmaceutically acceptable salt. In some embodiments, a salt can be prepared in situ during the final isolation and purification of a compound, or by separately reacting a purified compound in its free base form with a suitable organic or inorganic acid, and isolating the salt thus formed.

Examples of pharmaceutically acceptable salts can include those salts prepared by reaction of a compound disclosed herein with a mineral, organic acid or inorganic base, such salts can include, acetate, acrylate, adipate, alginate, aspartate, benzoate, benzenesulfonate, bisulfate, bisulfite, bitartrate, bromide, butyrate, butyn-1,4-dioate, camphorate, camphorsulfonate, caproate, caprylate, chlorobenzoate, chloride, citrate, cyclopentanepropionate, decanoate, digluconate, dihydrogenphosphate, dinitrobenzoate, dodecylsulfate, ethanesulfonate, formate, fumarate, glucoheptanoate, glycerophosphate, glycolate, hemisulfate, heptanoate, hexanoate, hexyne-1,6-dioate, hydroxybenzoate, γ-hydroxybutyrate, hydrochloride, hydrobromide, hydroiodide, 2-hydroxyethanesulfonate, iodide, isobutyrate, lactate, maleate, malonate, methanesulfonate, mandelate, metaphosphate, methanesulfonate, methoxybenzoate, methylbenzoate, monohydrogenphosphate, 1-napthalenesulfonate, 2-napthalenesulfonate, nicotinate, nitrate, palmoate, pectinate, persulfate, 3-phenylpropionate, phosphate, picrate, pivalate, propionate, pyrosulfate, pyrophosphate, propiolate, phthalate, phenylacetate, phenylbutyrate, propanesulfonate, salicylate, succinate, sulfate, sulfite, succinate, suberate, sebacate, sulfonate, tartrate, thiocyanate, tosylate, undeconate and xylenesulfonate.

The term “pharmaceutical composition,” or simply “composition” as used herein, can refer to an active agent, optionally mixed with at least one pharmaceutically acceptable chemical component, such as a carrier, a stabilizer, a diluent, a dispersing agent, a suspending agent, a thickening agent, an excipient, a bioadhesive, and the like. A dispensing agent can be an emulsion that can comprise a substantially uniform mixture of an organic phase and an aqueous phase. An organic phase and an aqueous phase can comprise components dissolved or suspended therein. While exemplary embodiments may describe a localization of a component with a single phase, it is understood that any component can be present in either phase.

The terms “co-administration”, “administered in combination with” and their grammatical equivalents or the like, as used herein, can encompass administration of selected therapeutic agents to a single patient, and can include treatment regimens in which the agents can be administered by the same or different route of administration or at the same or different times. In some embodiments, a compound disclosed herein can be co-administered with other agents. These terms can encompass administration of two or more agents to an animal so that both agents and/or their metabolites can be present in the animal at the same time. They can include simultaneous administration in separate compositions, administration at different times in separate compositions, and/or administration in a composition in which both agents can be present. Thus, in some embodiments, a compound and another agent(s) can be administered in a single composition. In some embodiments, a compound and another agent(s) can be admixed in the composition.

As used herein, the term “bioavailability” can denote the degree to which a drug such as an active agent, salt, metabolite, or other substance becomes available to the target tissue after administration.

Parameters often used in pharmacokinetic (PK) studies can include Tmax, Cmax, AUC(0−∞), AUC(0−t), and T_{1/2} and CL/F. “Tmax” can refer to the time to reach the maximal plasma concentration (“Cmax”) after administration of a therapeutic; “AUC(0−∞)” can refer to the area under the plasma concentration versus time curve from time 0 to infinity; “AUC(0−t)” can refer to the area under the plasma concentration versus time curve from time 0 to time t; “T_{1/2}” can refer to a half-life of a therapeutic in blood plasma; “T_{1/2, elim}” can refer to the half-life of elimination of the therapeutic from circulation; and “CL/F” can refer to an apparent clearance rate of a therapeutic.

The term “zero order release rate profile” or “zero order release profile” can refer to a profile in which a concentration of an active agent can be released into a vasculature of a subject at a constant rate over a given time interval. In some cases, an active agent can have a substantially zero order release profile into a serum, lymph, or peritoneal vasculature of a subject upon administering of the pharmaceutical composition.

The term “substantially free,” can be used to indicate certain ingredients that do not need to be included in a composition or mixture. The amount of ingredient can be so small that it does not cause irritation or generate an odor that could be objectionable to a subject. In some instances, the amount by weight of these ingredients can be less than about 5%, less than about 4.5%, less than about 4%, less than about 3.5%, less than about 3%, less than about 2.5%, less than about 2%, less than about 1.5%, less than about 1%, or less than about 0.5%. In some cases, the amount by weight of these ingredients can be less than about 0.9%, less than about 0.8%, less than about 0.7%, less than about 0.6%, less than about 0.5%, less than about 0.4%, less than about 0.3%, less than about 0.2%, or less than about 0.1%. In some cases, the amount by weight of these ingredients can be less than about 0.09%, less than about 0.08%, less than about 0.07%, less than about 0.06%, less than about 0.05%, less than about 0.04%, less than about 0.03%, less than about 0.02%, or less than about 0.01%. In some instances, the amount by weight of these ingredients can be from about 0.1% to about 5%, from about 0.1% to about 4.5%, from about 0.1% to about 4%, from about 0.1% to about 3.5%, from about 0.1% to about 3%, from about 0.1% to about 2.5%, from about 0.1% to about 2%, from about 0.1% to about 1.5%, from about 0.1% to about 1%, or from about 0.1% to about 0.5%. In some cases, the amount by weight of these ingredients can be 0%.

The term “bioadhesive” as used herein can refer to a polymeric material that can create an intimate contact between an active ingredient and a biological substrate. The term “mucoadhesion” can be used interchangeably to describe bioadhesion to a mucus membrane.

The term “w/w” as used herein can refer to a weight of a component of a composition relative to the total weight of a composition.

The term “emulsion” as used herein can refer to dispersion of two or more liquids. In some cases, an emulsion can include a dispersion of an organic phase in an aqueous phase. In some cases, an emulsion can include a dispersion of an aqueous phase in an organic phase. In some cases, an emulsion can include a dispersion of an organic phase in another organic phase. In some cases, an emulsion can include a dispersion of an aqueous phase in another aqueous phase. In some cases, an emulsion can be a uniform dispersion of the two or more liquids. In some cases, an emulsion can be a non-uniform dispersion of the two or more liquids.

The term “penetration enhancer” as used herein can refer to a compound added to a composition in order to enhance a rate of penetration of an active agent or salt thereof through a skin of a subject. Examples of penetration enhancers can include a sulfoxide (such as dimethylsulfoxide, DMSO), an Azone (such as laurocapram), a pyrrolidone (such as 2-pyrrolidone, 2P), a C₁-C₈ alcohol (such as methanol, ethanol, n-propanol, isopropanol, t-butanol, pentanol, hexanol, cyclohexanol, heptanol, octanol and the like), a glycol (such as propylene glycol), a surfactant, or a terpene.

The terms “dose” and “dosage” can be used interchangeably to refer to an amount of an active agent or a pharmaceutical composition administered to a subject.

### Formulation

Disclosed herein are pharmaceutical compositions for localized delivery of an active agent or a salt thereof. In some cases, a pharmaceutical composition can comprise an emulsion which can be a substantially uniform mixture of an organic phase and an aqueous phase, an active agent or a salt thereof, and a bioadhesive.

In some embodiments, a pharmaceutical composition can be formulated for vaginal delivery. Formulation of a pharmaceutical composition can include admixing an organic phase and aqueous phase with a bioadhesive and an active agent or salt thereof. In order to improve the delivery an active agent or a salt thereof, bioadhesives can be used to adhere a pharmaceutical composition to an epithelial surface upon administering vaginally of the pharmaceutical composition.

The inventors discovered the surprising and unexpected result that a pharmaceutical composition as described herein comprising emulsion can prevent or minimize vaginal clumping or discharge when formulated with a bioadhesive by emulsifying the insoluble material, thereby minimizing or eliminating unsightly clumping or discharge. Such an emulsion can comprise a substantially uniform mixture of an organic phase and an aqueous phase containing an active agent or salt thereof and a bioadhesive. It is envisaged that an aqueous phase and an organic phase can be provided as a uniform mixture, or that each component can be provided separately for mixture prior to an administering. A person of ordinary skill in the art would be capable of formulating either the uniform mixture or distinct phases depending on the application.

An emulsion containing an organic phase and an aqueous phase can contain various components or ingredients. In some cases, an organic phase can contain at least one oleogel. An oleogel can comprise at least one oily agent and at least one polymer.

An oily agent can be a monoglyceride, a diglyceride, a triglyceride, or any combination thereof. In some cases, an oily agent can be isolated and purified. In some cases, an oily agent can be a synthetic diglyceride, a synthetic triglyceride, a propylene glycol isostearate, a polyoxyethylenated oleic glyceride mixture, an oil of plant or natural origin, or any combination thereof.

In some cases, an oily agent can be present in a proportion of from about 0.1 to about 1%, 0.1 to about 2%, 0.1 to about 3%, 0.1 to about 4%, 0.1 to about 5%, 0.1 to about 6%, 0.1 to about 7%, 0.1 to about 8%, 0.1 to about 9%, 0.1 to about 10%, 0.1 to about 11%, 0.1 to about 12%, 0.1 to about 13%, 0.1 to about 14%, 0.1 to about 15%, 0.1 to about 16%, 0.1 to about 17%, 0.1 to about 18%, 0.1 to about 19%, 0.1 to about 20%, 0.1 to about 21%, 0.1 to about 22%, 0.1 to about 23%, 0.1 to about 24%, 0.1 to about 25%, 0.1 to about 26%, 0.1 to about 27%, 0.1 to about 28%, 0.1 to about 29%, or 0.1 to about 30% of the weight, relative to the weight of the organic phase.

In some cases, an oily agent can be present in a proportion of from about 0.01 to about 0.1%, 0.01 to about 0.2%, 0.01 to about 0.3%, 0.01 to about 0.4%, 0.01 to about 0.5%, 0.01 to about 0.6%, 0.01 to about 0.7%, 0.01 to about 0.8%, 0.01 to about 0.9%, 0.01 to about 1%, 0.01 to about 2%, 0.01 to about 3%, 0.01 to about 4%, 0.01 to about 5%, 0.01 to about 6%, 0.01 to about 7%, 0.01 to about 8%, 0.01 to about 9%, 0.01 to about 10%, 0.01 to about 11%, 0.01 to about 12%, 0.01 to about 13%, 0.01 to about 14%, 0.01 to about 15%, 0.01 to about 16%, 0.01 to about 17%, 0.01 to about 18%, 0.01 to about 19%, 0.01 to about 20%, 0.01 to about 21%, 0.01 to about 22%, 0.01 to about 23%, 0.01 to about 24%, 0.01 to about 25%, 0.01 to about 26%, 0.01 to about 27%, 0.01 to about 28%, 0.01 to about 29%, or 0.01 to about 30% of the weight, relative to the weight of the composition.

In some exemplary embodiments, an oily agent can be propylene glycol isostearate. Exemplary pharmaceutical compositions can comprise propylene glycol isostearate in a proportion of between of about 5 and 90% by weight, relative to the total weight of the oleogel.

A mono- di- or triglyceride can be a molecule of Formula I:

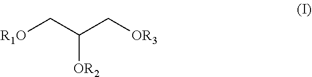

where R₁, R₂, and R₃ can independently be H; or C₁-C₂₀ alkyl comprising 0, 1, 2, 3, 4 or 5 degrees of unsaturation.

In some aspects, the synthetic mono- di- or triglyceride can be “LABRAFAC® lipophile WL1349”, sold by the company Gatefosse, propylene glycol isostearate, such as the product sold under the name “hydrophilol isostearique” by the company Gatefosse, and the polyglycolyzed glyceride “LABRRAFIL® M 1944 CS” as sold by Gatefosse.

LABRRAFIL® M 1944 CS is a mixture of polyoxyethylenated oleic glycerides obtained by the alcoholysis of natural plant oil. It can be an oily liquid whose properties are presented in table 1 below.

TABLE 1

| Chemical Name | Polyglycolyzed oleic glyceride |
|---|---|
| Trade name | LABRRAFIL ® M 1944 CS |
| Drop Point ° C. | Liq. |
| Saponification Number | 1451175 |
| Acid number | >2 |
| Iodine Number | 60/90 |
| Oral acute toxicity rat | OLD >20 mg/Kg |
| LOP | 0 |
| HLB | 314 |

In some cases, a mono-, di- or triglyceride can be of natural or plant origin. An oil of natural or plant origin can include an oil such as sweet almond oil, argan oil or palm oil.

A polymer in an organic phase can be a cellulose polymer. In some cases, a cellulose polymer can bean ethylcellulose, a non-sodium containing carboxymethylcellulose or a mixture thereof. In some cases, the polymer can be a water insoluble polymer. In some exemplary embodiments, a water insoluble polymer can be a water insoluble cellulose polymer.

A cellulose polymer can be a lipid soluble cellulose polymer. In some instance, the cellulose polymer can be an alkyl cellulose. In some instance, the alkyl cellulose can be methylcellulose, ethylcellulose, hydroxypropylcellulose or a combination thereof. In some instances, the cellulose polymer can be an alkyl carboxylic containing cellulose or a salt thereof. In some cases, the alkyl carboxylic containing cellulose can be non-sodium containing carboxymethylcellulose.

In some cases, the water insoluble polymer can be present in a proportion of from about 0.1 to about 1%, 0.1 to about 2%, 0.1 to about 3%, 0.1 to about 4%, 0.1 to about 5%, 0.1 to about 6%, 0.1 to about 7%, 0.1 to about 8%, 0.1 to about 9%, 0.1 to about 10%, 0.1 to about 11%, 0.1 to about 12%, 0.1 to about 13%, 0.1 to about 14%, 0.1 to about 15%, 0.1 to about 16%, 0.1 to about 17%, 0.1 to about 18%, 0.1 to about 19%, 0.1 to about 20%, 0.1 to about 21%, 0.1 to about 22%, 0.1 to about 23%, 0.1 to about 24%, 0.1 to about 25%, 0.1 to about 26%, 0.1 to about 27%, 0.1 to about 28%, 0.1 to about 29%, or 0.1 to about 30% of the weight, relative to the weight of the organic phase.

In some cases, the water insoluble polymer can be present in a proportion of from about 0.01 to about 0.1%, 0.01 to about 0.2%, 0.01 to about 0.3%, 0.01 to about 0.4%, 0.01 to about 0.5%, 0.01 to about 0.6%, 0.01 to about 0.7%, 0.01 to about 0.8%, 0.01 to about 0.9%, 0.01 to about 1%, 0.01 to about 2%, 0.01 to about 3%, 0.01 to about 4%, 0.01 to about 5%, 0.01 to about 6%, 0.01 to about 7%, 0.01 to about 8%, 0.01 to about 9%, 0.01 to about 10%, 0.01 to about 11%, 0.01 to about 12%, 0.01 to about 13%, 0.01 to about 14%, 0.01 to about 15%, 0.01 to about 16%, 0.01 to about 17%, 0.01 to about 18%, 0.01 to about 19%, 0.01 to about 20%, 0.01 to about 21%, 0.01 to about 22%, 0.01 to about 23%, 0.01 to about 24%, 0.01 to about 25%, 0.01 to about 26%, 0.01 to about 27%, 0.01 to about 28%, 0.01 to about 29%, or 0.01 to about 30% of the weight, relative to the weight of the composition.

In some exemplary embodiments, a cellulose polymer can be present in a proportion of 1 and about 10% by weight. In some exemplary embodiments, a cellulose polymer can be ethylcellulose present in a proportion of 1 and about 10% by weight based on a total weight of a composition. In some cases, an oily agent can comprise LABRRAFIL® M 1944 CS. In some cases, the oily agent can be present in a proportion of between about 5 and 90% by weight, relative to the total weight of the oleogel. In some cases, the ratio of the oleogel to the weight of the aqueous gel can be from about 10:90 to about 90:10. In some cases, the cellulose polymer can be EMULFREE® P. In some instances, the cellulose polymer can be EMULFREE® P and the oily agent can comprise LABRRAFIL® M 1944 CS.

An aqueous phase as described herein can comprise one or more aqueous gels. The aqueous phase can comprise at least one aqueous gel. In some cases, an aqueous gel can comprise at least one gelling agent. A gelling agent may be a carbomer, a poloxamer, a sodium carboxymethylcellulose, or any mixtures thereof.

In some cases, to gelling agent can be present in a proportion of from about 0.1 to about 1%, 0.1 to about 2%, 0.1 to about 3%, 0.1 to about 4%, 0.1 to about 5%, 0.1 to about 6%, 0.1 to about 7%, 0.1 to about 8%, 0.1 to about 9%, 0.1 to about 10%, 0.1 to about 11%, 0.1 to about 12%, 0.1 to about 13%, 0.1 to about 14%, 0.1 to about 15%, 0.1 to about 16%, 0.1 to about 17%, 0.1 to about 18%, 0.1 to about 19%, 0.1 to about 20%, 0.1 to about 21%, 0.1 to about 22%, 0.1 to about 23%, 0.1 to about 24%, 0.1 to about 25%, 0.1 to about 26%, 0.1 to about 27%, 0.1 to about 28%, 0.1 to about 29%, or 0.1 to about 30% of the weight, relative to the weight of the aqueous phase.

In some cases, a gelling agent can be present in a proportion of from about 0.01 to about 0.1%, 0.01 to about 0.2%, 0.01 to about 0.3%, 0.01 to about 0.4%, 0.01 to about 0.5%, 0.01 to about 0.6%, 0.01 to about 0.7%, 0.01 to about 0.8%, 0.01 to about 0.9%, 0.01 to about 1%, 0.01 to about 2%, 0.01 to about 3%, 0.01 to about 4%, 0.01 to about 5%, 0.01 to about 6%, 0.01 to about 7%, 0.01 to about 8%, 0.01 to about 9%, 0.01 to about 10%, 0.01 to about 11%, 0.01 to about 12%, 0.01 to about 13%, 0.01 to about 14%, 0.01 to about 15%, 0.01 to about 16%, 0.01 to about 17%, 0.01 to about 18%, 0.01 to about 19%, 0.01 to about 20%, 0.01 to about 21%, 0.01 to about 22%, 0.01 to about 23%, 0.01 to about 24%, 0.01 to about 25%, 0.01 to about 26%, 0.01 to about 27%, 0.01 to about 28%, 0.01 to about 29%, or 0.01 to about 30% of the weight, relative to the weight of the composition.

In some aspects, a gelling agent for the aqueous phase can be a carbomer, Carbopol 974 or Carbopol 980, present in a proportion of between of about 0.1 and about 5% by weight, relative to the total weight of the aqueous phase.

In some cases, the ratio of the weight of the organic phase to the weight of the aqueous phase can be from about 1:9 to about 9:1, from about 1:8 to about 8:1, from about 1:7 to about 7:1, from about 1:6 to about 6:1, from about 1:5 to about 5:1, from about 1:4 to about 4:1, from about 1:3 to about 3:1, from about 1:2 to about 2:1, or from about 1:1.5 to about 1.5:1. In some cases, the ratio of the weight of the organic phase to the weight of the aqueous phase can be about 1:1. In some aspects, an emulsion in the composition herein can comprise a substantially uniform mixture of an organic phase and an aqueous phase. In some cases, the ratio of the weight of the organic phase to the weight of the aqueous phase in such a substantially uniform mixture can be about 1:1.

In some cases, a uniform mixture of an organic phase and an aqueous phase can maintain a stable uniform appearance over a period of time when stored in a sealed container. In some cases, the mixture can be stable for at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, or 52 weeks. In some cases, the mixture can be stable for at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, months. In some cases, the mixture can be stable for at least about 1, 2, 3, 4, 5, 6, 7, 8, 9, or 10 years. In some cases, the sealed container can be stored at a temperature of about 25° C. at about 1 atm pressure. In some cases, the sealed container can be stored at about 25, 30, 35, 40, 45, 50, 55, 60, 65, 70, 75, 80, 85, 90, 95, 96, 97, 98, 99, or 100% relative humidity.

In some cases, the ratio of the volume of the organic phase to the volume of the aqueous phase can be from about 1:9 to about 9:1, from about 1:8 to about 8:1, from about 1:7 to about 7:1, from about 1:6 to about 6:1, from about 1:5 to about 5:1, from about 1:4 to about 4:1, from about 1:3 to about 3:1, from about 1:2 to about 2:1, or from about 1:1.5 to about 1.5:1. In some cases, the ratio of the volume of the organic phase to the volume of the aqueous phase can be about 1:1.

An emulsion can include dispersions or droplets, as well as other lipid structures that can form as a result of hydrophobic forces that drive a polar residue (e.g., long hydrocarbon chains) away from water and drive polar head groups toward water, when a water immiscible oily phase can be mixed with an aqueous phase. These other lipid structures can include unilamellar, paucilamellar, multilamellar lipid vesicles, micelles, and lamellar phases.

In some cases, a penetration enhancer can be present in an amount sufficient to achieve an enhanced rate of penetration. In some cases, a penetration enhancer does not enhance a rate of penetration of an active agent or salt thereof below a threshold concentration. A threshold concentration can be specific to a given penetration enhancer. In some cases, a penetration enhancer enhances a rate of absorption of an active agent or salt thereof when present at a concentration by weight of at least about 0.1%, 0.11%, 0.12%, 0.13%, 0.14%, 0.15%, 0.16%, 0.17%, 0.18%, 0.19%, 0.2%, 0.21%, 0.22%, 0.23%, 0.24%, 0.25%, 0.26%, 0.27%, 0.28%, 0.29%, 0.3%, 0.31%, 0.32%, 0.33%, 0.34%, 0.35%, 0.36%, 0.37%, 0.38%, 0.39%, 0.4%, 0.41%, 0.42%, 0.43%, 0.44%, 0.45%, 0.46%, 0.47%, 0.48%, 0.49%, 0.5%, 0.51%, 0.52%, 0.53%, 0.54%, 0.55%, 0.56%, 0.57%, 0.58%, 0.59%, 0.6%, 0.61%, 0.62%, 0.63%, 0.64%, 0.65%, 0.66%, 0.67%, 0.68%, 0.69%, 0.7%, 0.71%, 0.72%, 0.73%, 0.74%, 0.75%, 0.76%, 0.77%, 0.78%, 0.79%, 0.8%, 0.81%, 0.82%, 0.83%, 0.84%, 0.85%, 0.86%, 0.87%, 0.88%, 0.89%, 0.9%, 0.91%, 0.92%, 0.93%, 0.94%, 0.95%, 0.96%, 0.97%, 0.98%, 0.99%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, or 70% by weight relative to a total weight of a composition.

In some cases, a composition described herein does not contain a penetration enhancer. In some cases, a composition described herein contains no more than about 0.1%, 0.11%, 0.12%, 0.13%, 0.14%, 0.15%, 0.16%, 0.17%, 0.18%, 0.19%, 0.2%, 0.21%, 0.22%, 0.23%, 0.24%, 0.25%, 0.26%, 0.27%, 0.28%, 0.29%, 0.3%, 0.31%, 0.32%, 0.33%, 0.34%, 0.35%, 0.36%, 0.37%, 0.38%, 0.39%, 0.4%, 0.41%, 0.42%, 0.43%, 0.44%, 0.45%, 0.46%, 0.47%, 0.48%, 0.49%, 0.5%, 0.51%, 0.52%, 0.53%, 0.54%, 0.55%, 0.56%, 0.57%, 0.58%, 0.59%, 0.6%, 0.61%, 0.62%, 0.63%, 0.64%, 0.65%, 0.66%, 0.67%, 0.68%, 0.69%, 0.7%, 0.71%, 0.72%, 0.73%, 0.74%, 0.75%, 0.76%, 0.77%, 0.78%, 0.79%, 0.8%, 0.81%, 0.82%, 0.83%, 0.84%, 0.85%, 0.86%, 0.87%, 0.88%, 0.89%, 0.9%, 0.91%, 0.92%, 0.93%, 0.94%, 0.95%, 0.96%, 0.97%, 0.98%, 0.99%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, or 70% by weight of a penetration enhancer relative to a total weight of a composition.

The oleogel and the aqueous phase can respectively further comprise standard ingredients for a gel, such as texture agents, antioxidants, preservatives, dyes or fragrances of various types and in conventional amounts which are known to not cause skin irritation.

The composition can be in the form of a pharmaceutical composition. In some instances, the pharmaceutical composition can be administered similar to a cosmetic product in the form of a cream or gel that can be applied to the skin. In some instances, the composition can be in a unit dose form when applied to at least a portion of a skin in a specified amount. Since the pharmaceutical composition can comprise a stable mixture of an oleo-gel and an aqueous gel, it can be neither messy nor watery, and in comparison to conventional hydroalcoholic gels, it requires a smaller area for application, and can be quicker to dry.

In some instances, the gel composition can comprise a bioadhesive. Examples of bioadhesives can include carbomers, glyceryl monooleate, hypromellose, polycarbophil, poly(methylvinyl ether-co-maleic anhydride), as well as salts thereof. In some cases, a pharmaceutical composition can contain one or more bioadhesives. In some cases, a pharmaceutical composition can contain at least 1, 2, 3, 4, 5, 6, 7, 8, 9, or 10 bioadhesives.

In some exemplary embodiments, a bioadhesive can be polycarbophil or a salt thereof. Polycarbophil was designed to mimic negatively charged mucin, the glycoprotein component of mucus that is responsible for its attachment to underlying epithelial surfaces. Polycarbophil is a lightly cross-linked polymer. Polycarbophil is also a weak polyacid containing multiple carboxyl radicals (COO—) the source of its negative charges. These acid radicals can permit hydrogen bonding with a cell surface. Hydrogen bonds can be weak, in the case of polycarbophil they can be numerous. Bioadhesives such as polycarbophil can stay attached to vaginal epithelial cells until they turn over, which can be up to 7 days in menopausal women. However, vaginal clumping and discharge can occur due to the water insoluble polycarbophil remaining attached to the vaginal epithelial cells. Therefore there is a need to provide a formulation that can provide an adherence of a pharmaceutical composition to an epithelium of a subject while minimizing vaginal clumping or discharge.

In some cases, a bioadhesive can be present at a concentration by weight of at least about 0.01%, at least about 0.05%, at least about 0.1%, at least about 0.15%, at least about 0.2%, at least about 0.25%, at least about 0.3%, at least about 0.35%, at least about 0.4%, at least about 0.45%, at least about 0.5%, at least about 0.55%, at least about 0.6%, at least about 0.65%, at least about 0.7%, at least about 0.75%, at least about 0.8%, at least about 0.85%, at least about 0.9%, at least about 0.95%, at least about 1%, at least about 1.1%, at least about 1.2%, at least about 1.3%, at least about 1.4%, at least about 1.5%, at least about 1.6%, at least about 1.7%, at least about 1.8%, at least about 1.9%, at least about 2%, at least about 2.5%, at least about 3%, at least about 3.5%, at least about 4%, at least about 4.5%, at least about 5%, at least about 5.5%, at least about 6%, at least about 6.5%, at least about 7%, at least about 7.5%, at least about 8%, at least about 8.5%, at least about 9%, at least about 9.5%, or at least about 10%. In some instances, an aqueous gel can comprise a polycarbophil at a concentration by weight of from about 0.1% to about 10%, from about 0.1% to about 9%, from about 0.1% to about 8%, from about 0.1% to about 7%, from about 0.1% to about 6%, from about 0.1% to about 5%, from about 0.1% to about 4%, from about 0.1% to about 3%, from about 0.1% to about 2%, from about 0.1% to about 1%, from about 0.1% to about 0.9%, from about 0.1% to about 0.8%, from about 0.1% to about 0.7%, from about 0.1% to about 0.6%, from about 0.1% to about 0.5%, from about 0.1% to about 0.4%, from about 0.1% to about 0.3%, or from about 0.1% to about 0.2%.

The composition can comprise alcohol. For example, the alcohol can be a C₁-C₈ alcohol. Examples of a C₁-C₈ alcohol can include methanol, ethanol, n-propanol, isopropanol, t-butanol, pentanol, hexanol, cyclohexanol, heptanol, octanol and the like.

### Active Agents

A pharmaceutical composition as described herein can comprise at least one active agent or salt thereof. While exemplary active agents are described herein, it is possible substitute additional active agents in the composition in order to treat other indications treatable by administering of the pharmaceutical composition to a subject.

In some aspects, the active ingredients can be a hormone, an anti-inflammatory, an analgesic, a phenethylamine, an antineoplastic, a steroid, a 5-alpha reductase inhibitor, a gonadotropin-releasing hormone (GnRH) agonist, a GnRH antagonist, a glycine receptor antagonist, a tetrahydrocannabinol, an analgesic; an antibiotic, an antiviral compound, an antifungal compound, a salt of any of these, or any combination thereof.

In some instances, a hormone can be testosterone; dihydrotestosterone (DHT); estradiol; ethinylestradiol; progesterone; levonorgestrel; desogestrel; a peptide hormone such as oxytocin; a synthetic progesterone; a salt of any of these, or any combination thereof.

In some instances, an active ingredient can be an analgesic. Analgesics can include acetaminophen, bromfenac, diclofenac, diflunisal, etodolac, fenoprofen, flurbiprofen, ibuprofen, indomethacin, ketoprofen, ketorolac, meclofenamate, mefenamic acid, meloxicam, nabumetone, naproxen, nepafenac, oxaprozin, phenylbutazone, piroxicam, sulindac, tolmetin, celecoxib, buprenorphine, butorphanol, codeine, hydrocodone, hydromorphone, levorphanol, meperidine, methadone. Morphine, nalbuphine, oxycodone, oxymorphone, pentazocine, propoxyphene, tapentadol, tramadol, aspirin, a salt of any of these, or any combination thereof.

In some instances, a phenethylamine can be dopamine, epinephrine, norepinephrine, phenylephrine, methylphenidate, amphetamine, a salt of any of these, or any combination thereof.

In some instances, the antineoplastic can be cyclophosphamide, methotrexate, 5-fluorouracil, doxorubicin, procarbazine, prednisolone, bleomycin, vinblastine, dacarbazine, cisplatin, epirubicin, dichloroacetate, a salt of any of these, or any combination thereof.

In some instances, a steroid can be danazol or a salt thereof.

In some instances, a 5-alpha reductase inhibitor can be dutasteride, tamsulosin, finasteride a salt of any of these, or any combination thereof.

In some instances, a GnRH agonist, a GnRH antagonist, can be leuprolide, buserelin, histrelin, goserelin, deslorelin, nafarelin, triptorelin, cetrorelix, abarelix; ganirelix, ozarelix, degarelix or teverelix a salt of any of these, or any combination thereof.

In some instances, a GnRH antagonist can be cetrorelix, abarelix; ganirelix, ozarelix, degarelix, teverelix, a salt of any of these, or any combination thereof.

In some instances, a glycine receptor antagonist can be tranexamic acid or a salt thereof.

In some instances, an antibiotic can be Ceftobiprole; Ceftaroline; Clindamycin; Dalbavancin; Daptomycin; Linezolid; Mupirocin; Oritavancin; Tedizolid; Telavancin; Tigecycline; Vancomycin; an Aminoglycoside; a Carbapenem; Ceftazidime; Cefepime; Ceftobiprole; a Fluoroquinolone; Piperacillin; Ticarcillin; Linezolid; a Streptogramin; Tigecycline; Daptomycin; a salt of any of these; or any combination thereof

In some instances, an antiviral compound can be Ceftobiprole; Ceftaroline; Clindamycin; Dalbavancin; Daptomycin; Linezolid; Mupirocin; Oritavancin; Tedizolid; Telavancin; Tigecycline; Vancomycin; an Aminoglycoside; a Carbapenem; Ceftazidime; Cefepime; Ceftobiprole; a Fluoroquinolone; Piperacillin; Ticarcillin; Linezolid; a Streptogramin; Tigecycline; Daptomycin; a salt of any of these; or any combination thereof.

In some instances, an antifungal compound can be ciclopirox olamine; haloprogin; tolnaftate; undecylenate; topical nysatin; amorolfine; butenafine; naftifine; terbinafine; or any combination thereof.

In some aspects, a composition can comprise no more than about 10 mg, no more than about 20 mg, no more than about 40 mg, no more than about 60 mg, no more than about 80 mg, no more than about 100 mg, no more than about 120 mg, no more than about 140 mg, no more than about 160 mg, no more than about 180 mg, no more than about 200 mg, no more than about 300 mg, no more than about 400 mg, or no more than about 500 mg of an active agent or a salt thereof. In some cases, the composition can comprise at least about 10 mg, at least about 20 mg, at least about 40 mg, at least about 60 mg, at least about 80 mg, at least about 100 mg, at least about 120 mg, at least about 140 mg, at least about 160 mg, at least about 180 mg, at least about 200 mg, at least about 300 mg, at least about 400 mg, or at least about 500 mg of an active agent or a salt thereof.

An active agent can comprise a portion of the total weight of the composition. In some cases, an active agent can comprise from about 0.000001% to about 99%, from about 0.000001% to about 50%, from about 0.000001% to about 30%, from about 0.000001% to about 20%, from about 0.00005% to about 15%, from about 0.00005% to about 10%, from about 0.00001% to about 10%, from about 0.0001% to about 10%, or from about 0.0001% to about 5% by weight of the total weight of the composition.

In some instances, the composition can comprise alcohol at a concentration that allows the active agent to be solubilized. In some cases, the composition can have an alcohol concentration of at most about 10%, at most about 8%, at most about 6%, at most about 5%, at most about 4%, at most about 3%, at most about 2%, or at most about 1% by weight. In some cases, the alcohol concentration can be about 3.5% by weight.

In some instances, the composition can be substantially free of a surfactant. In some cases, a composition can comprise low amounts of surfactants. Surfactants can be non-ionic surfactants, cationic surfactants, amphoteric surfactants, or zwitterionic surfactants.

In some cases, a composition can have an surfactant concentration of at most about 10%, at most about 8%, at most about 6%, at most about 5%, at most about 4%, at most about 3%, at most about 2%, at most about 1%, or at most about 0.1% of a surfactant by weight of the total weight of the composition.

In the cases where a composition comprises an active agent or a salt thereof, the active agent or a salt thereof can be emulsified in the emulsion. When emulsified in the emulsion, the active agent can be in a form with an average particle size minimized to about the micrometer scale. In some instances, the average particle size can be minimized to about the nanometer scale. In some cases, the average particle size can be from about 0.001 nm to about 500 μm, from about 0.001 nm to about 400 μm, from about 0.001 nm to about 300 μm, from about 0.001 nm to about 200 μm, from about 0.001 nm to about 100 μm, from about 0.001 nm to about 90 μm, from about 0.001 nm to about 80 μm, from about 0.001 nm to about 70 μm, from about 0.001 nm to about 60 μm, from about 0.001 nm to about 50 μm, from about 0.001 nm to about 40 μm, from about 0.001 nm to about 30 μm, from about 0.001 nm to about 20 μm, from about 0.001 nm to about 10 μm, from about 0.001 nm to about 5 μm, from about 0.001 nm to about 1 μm, from about 0.001 nm to about 900 nm, from about 0.001 nm to about 800 nm, from about 0.001 nm to about 700 nm, from about 0.001 nm to about 600 nm, from about 0.001 nm to about 500 nm, from about 0.001 nm to about 400 nm, from about 0.001 nm to about 300 nm, from about 0.001 nm to about 200 nm, from about 0.001 nm to about 100 nm, from about 0.001 nm to about 90 nm, from about 0.001 nm to about 80 nm, from about 0.001 nm to about 70 nm, from about 0.001 nm to about 60 nm, from about 0.001 nm to about 50 nm, from about 0.001 nm to about 40 nm, from about 0.001 nm to about 30 nm, from about 0.001 nm to about 20 nm, from about 0.001 nm to about 10 nm, from about 0.001 nm to about 5 nm, from about 0.001 nm to about 1 nm, from about 0.001 nm to about 0.9 nm, from about 0.001 nm to about 0.8 nm, from about 0.001 nm to about 0.7 nm, from about 0.001 nm to about 0.6 nm, from about 0.001 nm to about 0.5 nm, from about 0.001 nm to about 0.4 nm, from about 0.001 nm to about 0.3 nm, from about 0.001 nm to about 0.2 nm, from about 0.001 nm to about 0.1 nm, from about 0.001 nm to about 0.09 nm, from about 0.001 nm to about 0.08 nm, from about 0.001 nm to about 0.07 nm, from about 0.001 nm to about 0.06 nm, from about 0.001 nm to about 0.05 nm, from about 0.001 nm to about 0.04 nm, from about 0.001 nm to about 0.03 nm, from about 0.001 nm to about 0.02 nm, or from about 0.001 nm to about 0.01 nm. In some instances, the average particle size can be about 0.01 nm, about 0.05 nm, about 0.1 nm, about 0.15 nm, about 0.2 nm, about 0.25 nm, about 0.3 nm, about 0.35 nm, about 0.4 nm, about 0.45 nm, about 0.5 nm, about 0.55 nm, about 0.6 nm, about 0.65 nm, about 0.7 nm, about 0.75 nm, about 0.8 nm, about 0.85 nm, about 0.9 nm, about 0.95 nm, about 1 nm, about 2 nm, about 3 nm, about 4 nm, about 5 nm, about 6 nm, about 7 nm, about 8 nm, about 9 nm, about 10 nm, about 15 nm, about 20 nm, about 25 nm, about 30 nm, about 35 nm, about 40 nm, about 45 nm, about 50 nm, about 55 nm, about 60 nm, about 65 nm, about 70 nm, about 75 nm, about 80 nm, about 85 nm, about 90 nm, about 95 nm, about 100 nm, about 150 nm, about 200 nm, about 250 nm, about 300 nm, about 350 nm, about 400 nm, about 450 nm, about 500 nm, about 550 nm, about 600 nm, about 650 nm, about 700 nm, about 750 nm, about 800 nm, about 850 nm, about 900 nm, about 950 nm, about 1 μm, about 2 μm, about 3 μm, about 4 μm, about 5 μm, about 6 μm, about 7 μm, about 8 μm, about 9 μm, about 10 μm, about 15 μm, about 20 μm, about 25 μm, about 30 μm, about 35 pm, about 40 μm, about 45 μm, about 50 μm, about 55 μm, about 60 μm, about 65 μm, about 70 pm, about 75 μm, about 80 μm, about 85 μm, about 90 μm, about 95 μm, about 100 μm, about 150 μm, about 200 μm, about 250 μm, about 300 μm, about 350 μm, about 400 μm, about 450 μm, or about 500 μm.

### Indications

The methods and compositions can be used for treating a condition or a disease. In some cases, a treating of a disease can include at least partially ameliorating at least one symptom of a disease or condition. Examples of a disease or condition that can be treated using a pharmaceutical composition as described herein can include an endometrial disorder, adenomyosis, a cancer, an inflammatory disorder, an infection, and any combination thereof. While exemplary diseases or conditions are recited herein, a person of ordinary skill in the art could use a pharmaceutical composition described herein to treat additional diseases or conditions by substituting additional active agents.

In some instances, a disease or condition can be an endometrial disorder. For example, an endometrial disorder may be endometriosis. Endometriosis can be an often painful disorder in which tissue that normally lines the inside of a uterus (e.g., the endometrium) grows outside the uterus (endometrial implant). The methods and compositions can be used for treating endometriosis that involves the ovaries, bowel or the tissue lining a pelvis or endometriosis in which the endometrial tissue spreads beyond a pelvic region. In endometriosis, displaced deposits break down and bleed with each menstrual cycle. Because this displaced tissue has no way to exit the body, it can become trapped. Surrounding tissue can become irritated, eventually developing scar tissue and adhesions—abnormal tissue that binds organs together. In some cases, treating the condition can comprise reducing an endometrial deposit to an amount that can be lower than prior to the treatment.

In some cases, an endometrial disorder can be adenomyosis. Adenomyosis is a disorder in which an inner lining of an endometrium breaks through the muscle wall of the uterus (the myometrium). Adenomyosis can cause menstrual cramps, lower abdominal pressure, and bloating before menstrual periods and can result in heavy periods. The condition can be located throughout the portions of a uterus or localized in one spot. Though the cause of adenomyosis isn't known, studies have suggested that various hormones—including estrogen, progesterone, prolactin, and follicle stimulating hormone—may trigger the condition. Current treatments can include an administering of an anti-inflammatory medication to reduce inflammation; administering of an intervention such as an aromatase inhibitor, a GnRH agonist, or a GnRH antagonist to suppress expression of hormones that can trigger the condition. In some cases, an incidence of adenomyosis and endometriosis can occur simultaneously.

Treatment of an endometrial disorder using a vaginal composition as described herein can be determined using a number of metrics known in the art. A treatment endpoint for an endometrial disorder can include an ability to become pregnant. In some cases, an ability to become pregnant can be determined using an in vitro assay, such as human chorionic gonadotropin (hCG) assay. An hCG assay can be performed on a sample from a subject, such as a blood sample. Such an assay could determine a level of hCG in the blood over time using an antibody specific for hCG, which can be used to determine an incidence of pregnancy. An ability to become pregnant can also be determined using an in vivo imaging means such as ultrasound to determine a presence of mature oocytes within the subject, or to determine a successful implantation of a zygote upon fertilization.

In some instances, a disease or condition can be a cancer. For example, the cancer can be cancer of the reproductive tract, cervical cancer, ovarian cancer, mesothelial cancer, peritoneal cancer, or any combination thereof.

In exemplary embodiments, administering vaginally a pharmaceutical composition described herein can be used to locally treat a cancer of the peritoneal cavity. Administering vaginally of a pharmaceutical composition described herein can be used to minimize adverse side effects that can occur through systemic administration. For example, a pharmaceutical composition comprising an antineoplastic as described above can be used to deliver the antineoplastic directly to the peritoneal cavity with only minimal delivery of the neoadjuvant into the circulatory system. As an exemplary illustration, such an administering vaginally of a pharmaceutical composition can be used to mitigate known side effects of systemic antineoplastic administration such as hair loss, reproductive side effects, skin or nail irritation, swelling, cardiotoxicity, hepatotoxicity, etc. Reduction of a side effect can be determined methods such as monitoring a lower incidence of a symptom of a side effect. Examples can include a reduction of local irritation or inflammation, a lower incidence of nausea, a lower incidence of pain, a reduction in irregular heart rate or arrhythmia, a decrease in overly frequent or painful urination, etc. Biomarkers can also be used to measure a decrease in a side effect. In some cases, a reduction of a side effect can include a reduction in a biomarker associated with toxicity. In some cases, a reduction of a side effect can include an increase in a biomarker associated with toxicity. Examples of biomarkers can include cardiac troponin I, cardiac troponin T, serum alanine aminotransferase, glutathione-S-transferase alpha, aspartate aminotransferase, serum creatinine, blood urea nitrogen, kidney injury molecule-1, eutrophil gelatinase-associated lipocalin (NGAL), interleukin-18 (IL-18), cystatin C, clusterin, fatty acid binding protein-liver type (L-FABP), osteopontin, etc.

In some cases, treating a cancer can comprise reducing reduce a tumor size, or slowing or preventing the growth of a tumor. A tumor burden of a subject can be determined using imaging techniques such as ultrasound or magnetic resonance imaging (MRI), which can be compared over time to determine a therapeutic effect of the administering of the pharmaceutical composition over time.

In some instances, the condition can be an inflammatory disorder. For example, the inflammatory disorder can be pelvic inflammatory disease, chromic pelvic pain, Other examples of inflammatory disease include sepsis, and chronic inflammation resulting from chronic viral or bacterial infection. In some cases, treating the condition can comprise reducing an amount of at least one proinflammatory cytokine to an amount that can be lower than prior to the treatment.

In some instances, the condition can be an infection. For example, the infection can be a bacterial infection, a viral infection, a fungal infection, or any combination thereof

In some cases, an infection can include infection by a bacterial pathogen. A bacterial pathogen may be derived from a bacterial species selected from the group, but not exclusive to the group, consisting of: Staphylococcus spp., e.g. Staphylococcus aureus (e.g. Staphylococcus aureus NCTC 10442 and Staphylococcus aureus ATCC25923), Staphylococcus epidermidis; Chlamydia spp., e.g. Chlamydia trachomatis, Chlamydia pneumoniae, Chlamydia psittaci; Enterococcus spp., e.g. Enterococcus faecalis; Streptococcus pyogenes; Listeria spp.; Pseudomonas spp.; Mycobacterium spp., e.g. Mycobacterium tuberculosis complex; Enterobacter spp.; Campylobacter spp.; Salmonella spp.; Streptococcus spp., e.g. Streptococcus Group A or B, Streptoccocus pneumoniae; Helicobacter spp., e.g. Helicobacter pylori, Helicobacter felis; Neisseria spp., e.g. Neisseria gonorrhoea, Neisseria meningitidis; Borrelia burgdorferi; Shigella spp., e.g. Shigella flexneri; Escherichia coli (E. coli 0157:H7 NCTC 12900); Haemophilus spp., e.g. Haemophilus influenzae; Francisella tularensis; Bacillus spp., e.g. Bacillus anthraces; Clostridia spp., e.g. Clostridium botulinum, Clostridium difficile; Yersinia spp., e.g. Yersinia pestis; Treponema spp.; Burkholderia spp., e.g. Burkholderia cepacia complex, B. mallei, B pseudomallei; Propionibacterium spp., e.g. P. acnes, Acinetobacter species, an Actinomyces species, a Campylobacter species, a Candida species, Corynebacterium minutissium, Corynebacterium pseudodiphtherias, Corynebacterium stratium, Corynebacterium group G1, Corynebacterium group G2, Enterobacteriaceae, an Enterococcus species, Klebsiella pneumoniae, a Moraxella species, a non-tuberculous mycobacteria species, a Porphyromonas species, Prevotella melaninogenicus, Salmonella typhimurium, Serratia marcescens Streptococcus agalactiae, Staphylococcus salivarius, Streptococcus mitis, Streptococcus sanguis, Streptococcus pneumoniae, Vibrio cholerae, a Coccidioides species, or a Cryptococcus species.

In some cases, an infection can include infection by a virus. A virus may be derived from the group, but not exclusive to the group, of a herpesvirus, a poxvirus, a hepadnavirus, a flavivirus, a togavirus, a coronavirus, hepatitis C, hepatitis D, an orthomyxovirus, a papillomavirus, a polyomaviridae, a parvovirus, a cytomegalovirus, an Epstein-Barr virus, a small pox virus, a cow pox virus, a sheep pox virus, an orf virus, a monkey pox virus, a vaccinia virus, a paramyxovirus, a retrovirus, an adenovirus, a rhabdovirus, a bunyavirus, a filovirus, an alphavirus, an arenavirus, a lentivirus, and any combination thereof. In some cases, the virus can be an enveloped virus. Examples of an enveloped viruses can include: a poxvirus, a hepadnavirus, a flavivirus, a togavirus, a coronavirus, hepatitis C, hepatitis D, an orthomyxovirus, a cytomegalovirus, an Epstein-Barr virus, a small pox virus, a cow pox virus, a sheep pox virus, an orf virus, a monkey pox virus, a vaccinia virus, a rhabdovirus, a bunyavirus, a filovirus, an alphavirus, an arenavirus, a lentivirus, and the like.

In some cases, an infection can include infection by a parasite selected from, but not limited to, the group consisting of Trypanosoma spp. (Trypanosoma cruzi, Trypansosoma brucei), Leishmania spp., Giardia spp., Trichomonas spp., Entamoeba spp., Naegleria spp., Acanthanioeba spp., Schistosoma spp., Plasmodium spp., Crytosporidium spp., Isospora spp., Balantidium spp., Loa Loa, Ascaris lumbricoides, Dirofilaria immitis, and Toxoplasma ssp., e.g. Toxoplasma gondii.

A fungal pathogen may be derived from a fungus (including yeast) selected from, but not limited to, the genera Candida spp., (e.g. C. albicans), Epidermophyton spp., Exophiala spp., Microsporum spp., Trichophyton spp., (e.g. T. rubrum and T. interdigitale), Tinea spp., Aspergillus spp., Blastomyces spp., Blastoschizomyces spp., Coccidioides spp., Cryptococcus spp. (e.g. Cryptococcus neoformans), Histoplasma spp., Paracoccidiomyces spp., Sporotrix spp., Absidia spp., Cladophialophora spp., Fonsecaea spp., Phialophora spp., Lacazia spp., Arthrographis spp., Acremoniwn spp., Actinomadura spp., Apophysomyces spp., Emmonsia spp., Basidiobolus spp., Beauveria spp., Chrysosporium spp., Conidiobolus spp., Cunninghamella spp., Fusarium spp., Geotrichum spp., Graphiwn spp., Leptosphaeria spp., Malassezia spp. (e.g. Malassezia Furfur), Mucor spp., Neotestudina spp., Nocardia spp., Nocardiopsis spp., Paecilomyces spp., Phoma spp., Piedraia spp., Pneunwcystis spp., Pseudallescheria spp., Pyrenochaeta spp., Rhizoinucor spp., Rhizopus spp., Rhodotorula spp., Saccharomyces spp., Scedosporium spp., Scopulariopsis spp., Sporobolomyces spp., S:yncephalastrum spp., Trichoderma spp., Trichosporon spp., Ulocladium spp., Ustilago spp., Verticillium spp., and Wangiella spp.

It is also envisaged that a pharmaceutical composition described herein could be administered prophylactically in order to prevent incidence of a disease or condition described herein. For example, a pharmaceutical composition comprising an antibiotic could be administered vaginally to a subject prior to surgery to prevent a peritoneal infection.

### Dosing/Pharmacokinetics

In some instances, a pharmaceutical formulation can be formulated to optimize pharmacokinetics/pharmacodynamics of an active agent or salt thereof contained therein upon administering vaginally of a pharmaceutical composition to a subject.

In some cases, a pharmaceutical composition comprising an active agent or salt thereof described herein can be administered at a dose of from about 1 mg to about 1000 mg, from about 5 mg to about 1000 mg, from about 10 mg to about 1000 mg, from about 15 mg to about 1000 mg, from about 20 mg to about 1000 mg, from about 25 mg to about 1000 mg, from about 30 mg to about 1000 mg, from about 35 mg to about 1000 mg, from about 40 mg to about 1000 mg, from about 45 mg to about 1000 mg, from about 50 mg to about 1000 mg, from about 55 mg to about 1000 mg, from about 60 mg to about 1000 mg, from about 65 mg to about 1000 mg, from about 70 mg to about 1000 mg, from about 75 mg to about 1000 mg, from about 80 mg to about 1000 mg, from about 85 mg to about 1000 mg, from about 90 mg to about 1000 mg, from about 95 mg to about 1000 mg, from about 100 mg to about 1000 mg, from about 150 mg to about 1000 mg, from about 200 mg to about 1000 mg, from about 250 mg to about 1000 mg, from about 300 mg to about 1000 mg, from about 350 mg to about 1000 mg, from about 400 mg to about 1000 mg, from about 450 mg to about 1000 mg, from about 500 mg to about 1000 mg, from about 550 mg to about 1000 mg, from about 600 mg to about 1000 mg, from about 650 mg to about 1000 mg, from about 700 mg to about 1000 mg, from about 750 mg to about 1000 mg, from about 800 mg to about 1000 mg, from about 850 mg to about 1000 mg, from about 900 mg to about 1000 mg, or from about 950 mg to about 1000 mg.

In some cases, a pharmaceutical composition comprising an active agent or salt thereof described herein can be administered at a dose of about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179 180, 181, 182, 183, 184, 184, 186, 187, 188, 189, 190, 191, 192, 193, 194, 195, 196, 197, 198, 199, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 mg.

In some cases, a pharmaceutical composition comprising an active agent or salt thereof described herein can be administered to provide a blood plasma concentration of an active agent, a metabolite thereof, or salt thereof of from about 0.5 ng/mL to about 10 ng/mL, from about 1 ng/mL to about 10 ng/mL, from about 5 ng/mL to about 10 ng/mL, from about 10 ng/mL to about 10 ng/mL, from about 15 ng/mL to about 10 ng/mL, from about 20 ng/mL to about 10 ng/mL, from about 25 ng/mL to about 10 ng/mL, from about 30 ng/mL to about 10 ng/mL, from about 35 ng/mL to about 10 ng/mL, from about 40 ng/mL to about 10 ng/mL, from about 45 ng/mL to about 10 ng/mL, from about 50 ng/mL to about 10 ng/mL, from about 55 ng/mL to about 10 ng/mL, from about 60 ng/mL to about 10 ng/mL, from about 65 ng/mL to about 10 ng/mL, from about 70 ng/mL to about 10 ng/mL, from about 75 ng/mL to about 10 ng/mL, from about 80 ng/mL to about 10 μg/mL, from about 85 ng/mL to about 10 μg/mL, from about 90 ng/mL to about 10 μg/mL, from about 95 ng/mL to about 10 μg/mL, from about 100 ng/mL to about 10 μg/mL, from about 200 ng/mL to about 10 μg/mL, from about 300 ng/mL to about 10 μg/mL, from about 400 ng/mL to about 10 μg/mL, from about 500 ng/mL to about 10 μg/mL, from about 600 ng/mL to about 10 μg/mL, from about 700 ng/mL to about 10 μg/mL, from about 800 ng/mL to about 10 μg/mL, from about 900 ng/mL to about 10 μg/mL, or from about 1 μg/mL to about 10 μg/mL after a time period of from about 1 minute to about 1, 2, 3, 4, 5, 6, 7, or 10 or more hours.

In some cases a pharmaceutical composition comprising an active agent or salt thereof described herein can be administered to provide a blood plasma concentration of an active agent, a metabolite thereof, or salt thereof after administration to a subject of at least about 200 ng/mL, 195 ng/mL, 190 ng/mL, 185 ng/mL, 180 ng/mL, 175 ng/mL, 170 ng/mL, 165 ng/mL, 160 ng/mL, 155 ng/mL, 150 ng/mL, 145 ng/mL, 140 ng/mL, 135 ng/mL, 130 ng/mL, 125 ng/mL, 120 ng/mL, 115 ng/mL, 110 ng/mL, 105 ng/mL, 100 ng/mL, 95 ng/mL, 90 ng/mL, 85 ng/mL, 80 ng/mL, 75 ng/mL, 70 ng/mL, 65 ng/mL, 60 ng/mL, 55 ng/mL, 50 ng/mL, 45 ng/mL, 40 ng/mL, 35 ng/mL, 30 ng/mL, 25 ng/mL, 20 ng/mL, 15 ng/mL, 10 ng/mL, or 5 ng/mL after a time period of from about 1 minute to about 1, 2, 3, 4, 5, 6 ,7, 8, 9, or 10 hours.

In some cases a pharmaceutical composition comprising an active agent or salt thereof described herein can be administered to provide a concentration of an active agent, a metabolite thereof, or salt thereof in a peritoneal fluid after administration to a subject of at least about 200 ng/mL, 195 ng/mL, 190 ng/mL, 185 ng/mL, 180 ng/mL, 175 ng/mL, 170 ng/mL, 165 ng/mL, 160 ng/mL, 155 ng/mL, 150 ng/mL, 145 ng/mL, 140 ng/mL, 135 ng/mL, 130 ng/mL, 125 ng/mL, 120 ng/mL, 115 ng/mL, 110 ng/mL, 105 ng/mL, 100 ng/mL, 95 ng/mL, 90 ng/mL, 85 ng/mL, 80 ng/mL, 75 ng/mL, 70 ng/mL, 65 ng/mL, 60 ng/mL, 55 ng/mL, 50 ng/mL, 45 ng/mL, 40 ng/mL, 35 ng/mL, 30 ng/mL, 25 ng/mL, 20 ng/mL, 15 ng/mL, 10 ng/mL, 9 ng/mL, 8 ng/mL, 7 ng/mL, 6 ng/mL, 5 ng/mL, 4 ng/mL, 3 ng/mL, 2 ng/mL, 1 ng/mL, 0.9 ng/mL, 0.8 ng/mL, 0.7 ng/mL, 0.6 ng/mL, 0.5 ng/mL, 0.4 ng/mL, 0.3 ng/mL, 0.2 ng/mL, or 0.1 ng/mL after a time period of from about 1 minute to about 1, 2, 3, 4, 5, 6 ,7, 8, 9, or 10 hours.

In some cases a pharmaceutical composition comprising an active agent or salt thereof described herein can be administered to provide a concentration of an active agent, a metabolite thereof, or salt thereof in a peritoneal fluid after administration to a subject of from about 0.5 ng/mL to about 100 ng/mL, from about 0.5 ng/mL to about 90 ng/mL, from about 0.5 ng/mL to about 80 ng/mL, from about 0.5 ng/mL to about 70 ng/mL, from about 0.5 ng/mL to about 60 ng/mL, from about 0.5 ng/mL to about 50 ng/mL, from about 0.5 ng/mL to about 40 ng/mL, from about 0.5 ng/mL to about 30 ng/mL, from about 0.5 ng/mL to about 20 ng/mL, from about 0.5 ng/mL to about 10 ng/mL, from about 0.5 ng/mL to about 9 ng/mL, from about 0.5 ng/mL to about 8 ng/mL, from about 0.5 ng/mL to about 7 ng/mL, from about 0.5 ng/mL to about 6 ng/mL, from about 0.5 ng/mL to about 5 ng/mL, from about 0.5 ng/mL to about 4 ng/mL, from about 0.5 ng/mL to about 3 ng/mL, from about 0.5 ng/mL to about 2 ng/mL, or from about 0.5 ng/mL to about 1 ng/mL after a time period of from about 1 minute to about 1, 2, 3, 4, 5, 6 ,7, 8, 9, or 10 hours.

In some cases a pharmaceutical composition comprising an active agent or salt thereof described herein can be administered to provide a concentration of an active agent, a metabolite thereof, or salt thereof in a peritoneal tissue after administration to a subject of at least about 200 ng/mg, 195 ng/mg, 190 ng/mg, 185 ng/mg, 180 ng/mg, 175 ng/mg, 170 ng/mg, 165 ng/mg, 160 ng/mg, 155 ng/mg, 150 ng/mg, 145 ng/mg, 140 ng/mg, 135 ng/mg, 130 ng/mg, 125 ng/mg, 120 ng/mg, 115 ng/mg, 110 ng/mg, 105 ng/mg, 100 ng/mg, 95 ng/mg, 90 ng/mg, 85 ng/mg, 80 ng/mg, 75 ng/mg, 70 ng/mg, 65 ng/mg, 60 ng/mg, 55 ng/mg, 50 ng/mg, 45 ng/mg, 40 ng/mg, 35 ng/mg, 30 ng/mg, 25 ng/mg, 20 ng/mg, 15 ng/mg, 10 ng/mg, 9 ng/mg, 8 ng/mg, 7 ng/mg, 6 ng/mg, 5 ng/mg, 4 ng/mg, 3 ng/mg, 2 ng/mg, 1 ng/mg, 0.9 ng/mg, 0.8 ng/mg, 0.7 ng/mg, 0.6 ng/mg, 0.5 ng/mg, 0.4 ng/mg, 0.3 ng/mg, 0.2 ng/mg, or 0.1 ng/mg after a time period of from about 1 minute to about 1, 2, 3, 4, 5, 6 ,7, 8, 9, or 10 hours.

In some cases a pharmaceutical composition comprising an active agent or salt thereof described herein can be administered to provide a concentration of an active agent, a metabolite thereof, or salt thereof in a peritoneal tissue after administration to a subject of from about 0.5 ng/mg to about 100 ng/mg, from about 0.5 ng/mg to about 90 ng/mg, from about 0.5 ng/mg to about 80 ng/mg, from about 0.5 ng/mg to about 70 ng/mg, from about 0.5 ng/mg to about 60 ng/mg, from about 0.5 ng/mg to about 50 ng/mg, from about 0.5 ng/mg to about 40 ng/mg, from about 0.5 ng/mg to about 30 ng/mg, from about 0.5 ng/mg to about 20 ng/mg, from about 0.5 ng/mg to about 10 ng/mg, from about 0.5 ng/mg to about 9 ng/mg, from about 0.5 ng/mg to about 8 ng/mg, from about 0.5 ng/mg to about 7 ng/mg, from about 0.5 ng/mg to about 6 ng/mg, from about 0.5 ng/mg to about 5 ng/mg, from about 0.5 ng/mg to about 4 ng/mg, from about 0.5 ng/mg to about 3 ng/mg, from about 0.5 ng/mg to about 2 ng/mg, or from about 0.5 ng/mg to about 1 ng/mg after a time period of from about 1 minute to about 1, 2, 3, 4, 5, 6 ,7, 8, 9, or 10 hours.

In some cases, a pharmaceutical composition comprising an active agent or salt thereof described herein can be administered to provide a Tmax of an active agent or salt thereof after administration to a subject of from about 1 minute to about 600 minutes, from about 1 minute to about 590 minutes, from about 1 minute to about 580 minutes, from about 1 minute to about 570 minutes, from about 1 minute to about 560 minutes, from about 1 minute to about 550 minutes, from about 1 minute to about 540 minutes, from about 1 minute to about 530 minutes, from about 1 minute to about 520 minutes, from about 1 minute to about 510 minutes, from about 1 minute to about 500 minutes, from about 1 minute to about 490 minutes, from about 1 minute to about 480 minutes, from about 1 minute to about 470 minutes, from about 1 minute to about 460 minutes, from about 1 minute to about 450 minutes, from about 1 minute to about 440 minutes, from about 1 minute to about 430 minutes, from about 1 minute to about 420 minutes, from about 1 minute to about 410 minutes, from about 1 minute to about 400 minutes, from about 1 minute to about 390 minutes, from about 1 minute to about 380 minutes, from about 1 minute to about 370 minutes, from about 1 minute to about 360 minutes, from about 1 minute to about 350 minutes, from about 1 minute to about 340 minutes, from about 1 minute to about 330 minutes, from about 1 minute to about 320 minutes, from about 1 minute to about 310 minutes, from about 1 minute to about 300 minutes, from about 1 minute to about 290 minutes, from about 1 minute to about 280 minutes, from about 1 minute to about 270 minutes, from about 1 minute to about 260 minutes, from about 1 minute to about 250 minutes, from about 1 minute to about 240 minutes, from about 1 minute to about 230 minutes, from about 1 minute to about 220 minutes, from about 1 minute to about 210 minutes, from about 1 minute to about 200 minutes, from about 1 minute to about 190 minutes, from about 1 minute to about 180 minutes, from about 1 minute to about 170 minutes, from about 1 minute to about 160 minutes, from about 1 minute to about 150 minutes, from about 1 minute to about 140 minutes, from about 1 minute to about 130 minutes, from about 1 minute to about 120 minutes, from about 1 minute to about 110 minutes, from about 1 minute to about 100 minutes, from about 1 minute to about 90 minutes, from about 1 minute to about 80 minutes, from about 1 minute to about 70 minutes, from about 1 minute to about 60 minutes, from about 1 minute to about 50 minutes, from about 1 minute to about 40 minutes, from about 1 minute to about 30 minutes, from about 1 minute to about 20 minutes, from about 1 minute to about 10 minutes, from about 1 minute to about 9 minutes, from about 1 minute to about 8 minutes, from about 1 minute to about 7 minutes, from about 1 minute to about 6 minutes, from about 1 minute to about 5 minutes, from about 1 minute to about 4 minutes, from about 1 minute to about 3 minutes, or from about 1 minute to about 2 minutes.

In some cases, a pharmaceutical composition comprising an active agent or salt thereof. described herein can be administered to provide a Tmax of an active agent, a metabolite thereof, or salt thereof after administration to a subject of at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179 180, 181, 182, 183, 184, 184, 186, 187, 188, 189, 190, 191, 192, 193, 194, 195, 196, 197, 198, 199, or 200 minutes. In some cases, a pharmaceutical composition comprising an active agent or salt thereof described herein can be administered to provide a Tmax of an active agent, a metabolite thereof, or salt thereof after administration to a subject of at least about 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, or 7.0 hours.

In some cases, a pharmaceutical composition comprising an active agent or salt thereof described herein can be administered to provide a Cmax of an active agent, a metabolite thereof, or salt thereof after administration to a subject of at least about 1,000 μg/mL, 950 μg/mL, 900 μg/mL, 850 μg/mL, 800 μg/mL, 750 μg/mL, 700 μg/mL, 650 μg/mL, 600 μg/mL, 550 μg/mL, 500 μg/mL, 450 μg/mL, 400 μg/mL, 350 μg/mL, 300 μg/mL, 250 μg/mL, 200 μg/mL, 150 μg/mL, 100 μg/mL, or 50 μg/mL. In some cases, a pharmaceutical composition comprising an active agent or salt thereof described herein can be administered to provide a Cmax of an active agent, a metabolite thereof, or salt thereof after administration to a subject of at least about 100 μg/mL, 95 μg/mL, 90 μg/mL, 85 μg/mL, 80 μg/mL, 75 μg/mL, 70 μg/mL, 65 μg/mL, 60 μg/mL, 55 μg/mL, 50 μg/mL, 45 μg/mL, 40 μg/mL, 35 μg/mL, 30 μg/mL, 25 μg/mL, 20 μg/mL, 15 μg/mL, 10 μg/mL, 5 μg/mL, 4 μg/mL, 3 μg/mL, 2 μg/mL, or 1 μg/mL. In some cases, an active agent, salt thereof, or pharmaceutical composition comprising an active agent or salt thereof described herein can be administered to provide a Cmax of an active agent, a metabolite thereof, or salt thereof after administration to a subject of at least about 1,000 ng/mL, 950 ng/mL, 900 ng/mL, 850 ng/mL, 800 ng/mL, 750 ng/mL, 700 ng/mL, 650 ng/mL, 600 ng/mL, 550 ng/mL, 500 ng/mL, 450 ng/mL, 400 ng/mL, 350 ng/mL, 300 ng/mL, 250 ng/mL, 200 ng/mL, 150 ng/mL, 100 ng/mL, or 50 ng/mL. In some cases, a pharmaceutical composition comprising an active agent or salt thereof described herein can be administered to provide a Cmax of an active agent, a metabolite thereof, or salt thereof after administration to a subject of at least about 100 ng/mL, 95 ng/mL, 90 ng/mL, 85 ng/mL, 80 ng/mL, 75 ng/mL, 70 ng/mL, 65 ng/mL, 60 ng/mL, 55 ng/mL, 50 ng/mL, 45 ng/mL, 40 ng/mL, 35 ng/mL, 30 ng/mL, 25 ng/mL, 20 ng/mL, 15 ng/mL, 10 ng/mL, or 5 ng/mL. In some cases, a pharmaceutical composition comprising an active agent or salt thereof described herein can be administered to provide a Cmax of an active agent, a metabolite thereof, or salt thereof of at least about 50 ng/mL, 49 ng/mL, 48 ng/mL, 47 ng/mL, 46 ng/mL, 45 ng/mL, 44 ng/mL, 43 ng/mL, 42 ng/mL, 41 ng/mL, 40 ng/mL, 39 ng/mL, 38 ng/mL, 37 ng/mL, 36 ng/mL, 35 ng/mL, 34 ng/mL, 33 ng/mL, 32 ng/mL, 31 ng/mL, 30 ng/mL, 29 ng/mL, 28 ng/mL, 27 ng/mL, 26 ng/mL, 25 ng/mL, 24 ng/mL, 23 ng/mL, 22 ng/mL, 21 ng/mL, 20 ng/mL, 19 ng/mL, 18 ng/mL, 17 ng/mL, 16 ng/mL, 15 ng/mL, 14 ng/mL, 13 ng/mL, 12 ng/mL, 11 ng/mL, 10 ng/mL, 9 ng/mL, 8 ng/mL, 7 ng/mL, 6 ng/mL, 5 ng/mL, 4 ng/mL, 3 ng/mL, 2 ng/mL, 1 ng/mL, or 0.5 ng/mL.

In some instances, a pharmaceutical composition comprising an active agent or salt thereof described herein can be administered to provide an AUC(0−t) of an active agent, a metabolite thereof, or salt thereof after administration to a subject of at least about 10,000 ng*h/mL, 9,900 ng*h/mL, 9,800 ng*h/mL, 9,700 ng*h/mL, 9,600 ng*h/mL, 9,500 ng*h/mL, 9,400 ng*h/mL, 9,300 ng*h/mL, 9,200 ng*h/mL, 9,100 ng*h/mL, 9,000 ng*h/mL, 8,900 ng*h/mL, 8,800 ng*h/mL, 8,700 ng*h/mL, 8,600 ng*h/mL, 8,500 ng*h/mL, 8,400 ng*h/mL, 8,300 ng*h/mL, 8,200 ng*h/mL, 8,100 ng*h/mL, 8,000 ng*h/mL, 7,900 ng*h/mL, 7,800 ng*h/mL, 7,700 ng*h/mL, 7,600 ng*h/mL, 7,500 ng*h/mL, 7,400 ng*h/mL, 7,300 ng*h/mL, 7,200 ng*h/mL, 7,100 ng*h/mL, 7,000 ng*h/mL, 6,900 ng*h/mL, 6,800 ng*h/mL, 6,700 ng*h/mL, 6,600 ng*h/mL, 6,500 ng*h/mL, 6,400 ng*h/mL, 6,300 ng*h/mL, 6,200 ng*h/mL, 6,100 ng*h/mL, 6,000 ng*h/mL, 5,900 ng*h/mL, 5,800 ng*h/mL, 5,700 ng*h/mL, 5,600 ng*h/mL, 5,500 ng*h/mL, 5,400 ng*h/mL, 5,300 ng*h/mL, 5,200 ng*h/mL, 5,100 ng*h/mL, 5,000 ng*h/mL, 4,500 ng*h/mL, 4,000 ng*h/mL, 3,500 ng*h/mL, 3,000 ng*h/mL, 2,500 ng*h/mL, 2,000 ng*h/mL, 1,500 ng*h/mL, or 1,900 ng*h/mL, where t can be at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 hours after administration of a pharmaceutical composition comprising an active agent or salt thereof.

In some instances, a pharmaceutical composition comprising an active agent or salt thereof described herein can be administered to provide an AUC(0−t) of a an active agent, a metabolite thereof, or salt thereof after administration to a subject of at least about 10,000 ng*h/mL, 9,900 ng*h/mL, 9,800 ng*h/mL, 9,700 ng*h/mL, 9,600 ng*h/mL, 9,500 ng*h/mL, 9,400 ng*h/mL, 9,300 ng*h/mL, 9,200 ng*h/mL, 9,100 ng*h/mL, 9,000 ng*h/mL, 8,900 ng*h/mL, 8,800 ng*h/mL, 8,700 ng*h/mL, 8,600 ng*h/mL, 8,500 ng*h/mL, 8,400 ng*h/mL, 8,300 ng*h/mL, 8,200 ng*h/mL, 8,100 ng*h/mL, 8,000 ng*h/mL, 7,900 ng*h/mL, 7,800 ng*h/mL, 7,700 ng*h/mL, 7,600 ng*h/mL, 7,500 ng*h/mL, 7,400 ng*h/mL, 7,300 ng*h/mL, 7,200 ng*h/mL, 7,100 ng*h/mL, 7,000 ng*h/mL, 6,900 ng*h/mL, 6,800 ng*h/mL, 6,700 ng*h/mL, 6,600 ng*h/mL, 6,500 ng*h/mL, 6,400 ng*h/mL, 6,300 ng*h/mL, 6,200 ng*h/mL, 6,100 ng*h/mL, 6,000 ng*h/mL, 5,900 ng*h/mL, 5,800 ng*h/mL, 5,700 ng*h/mL, 5,600 ng*h/mL, 5,500 ng*h/mL, 5,400 ng*h/mL, 5,300 ng*h/mL, 5,200 ng*h/mL, 5,100 ng*h/mL, 5,000 ng*h/mL, 4,500 ng*h/mL, 4,000 ng*h/mL, 3,500 ng*h/mL, 3,000 ng*h/mL, 2,500 ng*h/mL, 2,000 ng*h/mL, 1,500 ng*h/mL, or 1,900 ng*h/mL, where t can be at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 days after administration of a pharmaceutical composition comprising an active agent or salt thereof.

In some exemplary embodiments, a pharmaceutical composition comprising an active agent or salt thereof described herein can be administered to provide an AUC(0−t) of an active agent, a metabolite thereof, or salt thereof after administration to a subject of from about 1,000 ng*h/mL to about 10,000 ng*h/mL, from about 1,000 ng*h/mL to about 9,000 ng*h/mL, from about 1,000 ng*h/mL to about 8,000 ng*h/mL, from about 1,000 ng*h/mL to about 7,000 ng*h/mL, from about 1,000 ng*h/mL to about 6,000 ng*h/mL, from about 1,000 ng*h/mL to about 5,000 ng*h/mL, from about 1,000 ng*h/mL to about 4,000 ng*h/mL, from about 1,000 ng*h/mL to about 3,000 ng*h/mL, or from about 1,000 ng*h/mL to about 2,000 ng*h/mL, where t can be at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 days after administration of a pharmaceutical composition comprising an active agent or salt thereof.

In some exemplary embodiments, a pharmaceutical formulation can be produced such that when a pharmaceutical formulation is administered to a primate, an active agent or salt thereof can have a Tₘₐₓ of from about 1 minute to about 1 hour, a Cₘₐₓ of from about 1 minute to about 8 hours, an AUC_{0>24 hour} of from about 0.1 μg·hr/L to about 1,000 μg·hr/L, a half-life of from about 2 hours to about 24 hours, or a combination thereof.

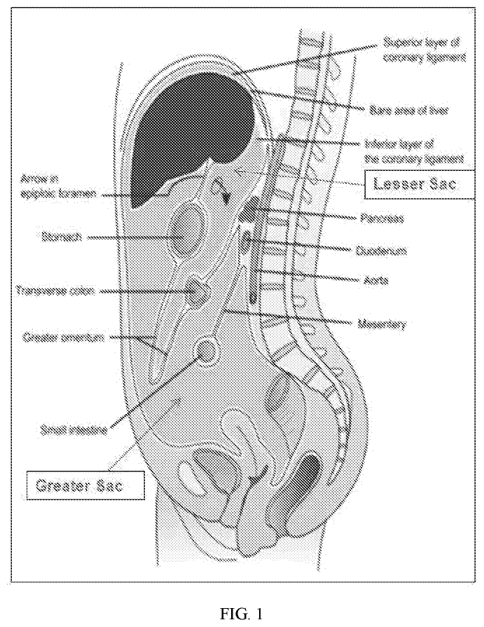

In some instances, a pharmaceutical formulation can be formulated such that, when a pharmaceutical formulation is administered to a subject, an active agent or salt thereof can be substantially localized in a peritoneal organ or tissue of a subject. Examples of organs or tissues of the peritoneal cavity can include those depicted in FIG. 1. A peritoneal cavity can include peritoneal fluid. A peritoneal organ or tissue can include, but is not limited to: a bladder, a gall bladder, an intestine, a uterus, an endometrium, a myometrium, a perimetrium, a stomach, an ovary, an ovarian cortex, an ovarian epithelium, a liver, a spleen, or a kidney.

In some instances, when a pharmaceutical formulation is administered to a subject, an active agent can have a half-life of about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179 180, 181, 182, 183, 184, 184, 186, 187, 188, 189, 190, 191, 192, 193, 194, 195, 196, 197, 198, 199, or 200 minutes. In some instances, when a pharmaceutical formulation is administered to a subject, an active agent or salt thereof can have a half-life of about 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, or 7.0 hours. In some instances, when a pharmaceutical formulation is administered to a subject, an active agent or salt thereof can have a half-life of from about 1 minute to about 600 minutes, from about 1 minute to about 590 minutes, from about 1 minute to about 580 minutes, from about 1 minute to about 570 minutes, from about 1 minute to about 560 minutes, from about 1 minute to about 550 minutes, from about 1 minute to about 540 minutes, from about 1 minute to about 530 minutes, from about 1 minute to about 520 minutes, from about 1 minute to about 510 minutes, from about 1 minute to about 500 minutes, from about 1 minute to about 490 minutes, from about 1 minute to about 480 minutes, from about 1 minute to about 470 minutes, from about 1 minute to about 460 minutes, from about 1 minute to about 450 minutes, from about 1 minute to about 440 minutes, from about 1 minute to about 430 minutes, from about 1 minute to about 420 minutes, from about 1 minute to about 410 minutes, from about 1 minute to about 400 minutes, from about 1 minute to about 390 minutes, from about 1 minute to about 380 minutes, from about 1 minute to about 370 minutes, from about 1 minute to about 360 minutes, from about 1 minute to about 350 minutes, from about 1 minute to about 340 minutes, from about 1 minute to about 330 minutes, from about 1 minute to about 320 minutes, from about 1 minute to about 310 minutes, from about 1 minute to about 300 minutes, from about 1 minute to about 290 minutes, from about 1 minute to about 280 minutes, from about 1 minute to about 270 minutes, from about 1 minute to about 260 minutes, from about 1 minute to about 250 minutes, from about 1 minute to about 240 minutes, from about 1 minute to about 230 minutes, from about 1 minute to about 220 minutes, from about 1 minute to about 210 minutes, from about 1 minute to about 200 minutes, from about 1 minute to about 190 minutes, from about 1 minute to about 180 minutes, from about 1 minute to about 170 minutes, from about 1 minute to about 160 minutes, from about 1 minute to about 150 minutes, from about 1 minute to about 140 minutes, from about 1 minute to about 130 minutes, from about 1 minute to about 120 minutes, from about 1 minute to about 110 minutes, from about 1 minute to about 100 minutes, from about 1 minute to about 90 minutes, from about 1 minute to about 80 minutes, from about 1 minute to about 70 minutes, from about 1 minute to about 60 minutes, from about 1 minute to about 50 minutes, from about 1 minute to about 40 minutes, from about 1 minute to about 30 minutes, from about 1 minute to about 20 minutes, from about 1 minute to about 10 minutes, from about 1 minute to about 9 minutes, from about 1 minute to about 8 minutes, from about 1 minute to about 7 minutes, from about 1 minute to about 6 minutes, from about 1 minute to about 5 minutes, from about 1 minute to about 4 minutes, from about 1 minute to about 3 minutes, or from about 1 minute to about 2 minutes.

### Methods of Detection

Also disclosed herein are methods of detecting an active agent, a metabolite thereof, or a salt thereof in a sample from a subject after an administering of a pharmaceutical composition to a subject. A sample from a subject may be blood or any excretory liquid. Non-limiting examples of may include saliva, blood, serum, cerebrospinal fluid, semen, feces, plasma, or urine.

In some exemplary embodiments, the method can be a method of detecting danazol or a salt thereof in a sample from a subject. A method can comprise contacting a portion of a sample from a subject with an albumin; and detecting danazol or a salt thereof by mass spectrometry. An albumin can include a human or bovine albumin. In some cases, the albumin can be a serum albumin.

Detection of an active agent such as danazol can be carried out by contacting the active agent with an albumin to form an albumin adduct and determining a presence of the adduct using mass spectrometry. A method can further comprise comparison to an internal standard. An exemplary internal standard can include 19-Norethindrone.

A mass spectrometer can include a single or tandem mass spectrometer. A mass spectrometer can comprise an electrospray ionizer, a matrix-assisted laser desorption/ionization ionizer, an electron ionizer, a fast atom bombardment ionizer or a chemical ionizer.

An exemplary method can comprise detecting danazol by determining an amount of an [M+H]⁺ ion. In some cases, an [M+H]⁺ ion can comprise an m/z of about 338.

In some instances, a sample can be dried after contacting with an albumin. In some instances, a sample can be reconstituted, resuspended, or diluted in a resuspension buffer after contacting with an albumin. A resuspension buffer can a buffering agent such as saline, citrate, phosphate, phosphate buffered saline, acetate, glycine, tris(hydroxymethyl)aminomethane (tris) hydrochloride, tris buffered saline (TBS), 3-[[1,3-dihydroxy-2-(hydroxymethyl)propan-2-yl]amino]propane-1-sulfonic acid (TAPS), bicine, tricine, 3-[[1,3-dihydroxy-2-(hydroxymethyl)propan-2-yl]amino]-2-hydroxypropane-1-sulfonic acid (TAPSO), 4-(2-hydroxyethyl)-1-piperazineethanesulfonic acid (HEPES), piperazine-N,N′-bis(2-ethanesulfonic acid) (PIPES), 3-(N-morpholino)propanesulfonic acid (MOPS), 2-(N-morpholino)ethanesulfonic acid (MES), 2-[[1,3-dihydroxy-2-(hydroxymethyl)propan-2-yl]amino]ethanesulfonic acid (TES), cacodylate, glycine, carbonate, or any combination thereof.

### Kits

Disclosed herein are kits. In some aspects, a composition can be packaged in a container. In some aspects, a kit can further comprise instructions that direct administration of a unit dose of a composition to a subject.

Methods of making a kit can include placing a pharmaceutical composition in a container. A method can further comprise an inclusion of instructions for use. In some cases, instructions for use can direct administration of a unit dose of a composition to a subject.

Also disclosed herein are kits for determining an amount of danazol in a sample. A kit can comprise: a sample collection vessel; an albumin; and instructions for use. In some cases, an internal standard can be 19-Norethindrone or a salt thereof. Instructions for use can direct a user to: collect a sample in the sample collection vessel; contact a portion of the sample with an amount of the albumin; and detect danazol or a salt thereof by mass spectrometry. In some cases, a kit can further comprise an internal standard. An internal standard can be 19-Norethindrone or a salt thereof.

### EXAMPLES

### Example 1: Formulation of a Composition Comprising a Hormone of Vaginal Delivery

An exemplary pharmaceutical composition containing a bioadhesive for delivering a hormone can be prepared for administering vaginally to a subject using the following formulation:

### Progesterone Formulation (% w/w)

| | |
|---|---|
| Micronized Progesterone (USP/EP) | 8.0% |
| Carbomer (Carbopol 974P NF) | 1.0% |
| Polycarbophil Noveon AA1 (USP) | 1.5% |
| Sorbic Acid (USP/EP) | 0.1% |
| Labrafac Lipophile WL 1349 (USP/EP) | 17.07% |
| EmulFree P Pharma Grade | 2.0% |
| Purified Water (USP/EP) | 68.4% |
| Sodium Hydroxide (USP/EP) QS AD pH 2.8-3.2 | |
| Purified Water (USP/EP) | QS 100% |

The progesterone formulation can be manufactured by dissolving micronized progesterone in an oil phase consisting of LABRAFAC® lipophille WL 1349 and EMULFREE® P. To this suspension, alcohol can be added at a concentration in which the alcohol does not behave as a penetration enhancer. The carbomers will be hydrated in the aqueous phase. After hydration, sorbic acid, polycarbophil, and sodium hydroxide will be added to the aqueous phase. The two phases will be mixed in an appropriate vessel. The provided composition will be stored in a sealed container prior administering vaginally.

In addition to progesterone P(4) any synthetic progestin could be formulated utilizing described formulation.

### Estradiol Formulation: (% w/w)

| | |
|---|---|
| Micronized Estradiol (USP/EP) | 0.1% |
| Carbomer (Carbopol 974P NF) | 1.0% |
| Polycarbophil Noveon AA1 (USP) | 1.5% |
| Sorbic Acid (USP/EP) | 0.1% |
| Labrafac Lipophile WL 1349 (USP/EP) | 17.07% |
| EmulFree P Pharma Grade | 2.0% |
| Purified Water (USP/EP) | 76.4% |
| Sodium Hydroxide (USP/EP) QS AD pH 2.8-3.2 | |
| Purified Water (USP/EP) | QS 100% |

Any estrogen could be substituted for estradiol for example, dienestrol, estriol, estrone etc.

Compositions containing a bioadhesive and other active ingredients can be prepared in a similar fashion to the formulations described above Other active ingredients can include other hormones; antineoplastics; receptor agonists or antagonists; steroids; antibiotics; antiviral compounds; antifungal compounds; and anti-inflammatories.

### Example 2: Formulation of a Composition Comprising Danazol for Vaginal Delivery

An exemplary pharmaceutical composition containing a bioadhesive for delivering danazol can be prepared for administering vaginally to a subject using the following formulation:

### Danazol Formulation: (% w/w)

| | |
|---|---|
| Danazol | 6.67% |
| Carbomer (Carbopol 974P NF) | 1.0% |
| Polycarbophil Noveon AA1 (USP) | 1.5% |
| Methylparaben | 0.25% |
| Labrafac Lipophile WL 1349 (USP/EP) | 15% |
| EmulFree P Pharma Grade | 2.0% |
| Purified Water (USP/EP) | 73.58% |

The danazol formulation can be manufactured by dissolving danazol in an oil phase consisting of LABRAFAC® lipophille WL 1349 and EMULFREE® P. The carbomers will be hydrated in the aqueous phase. After hydration, methylparaben and polycarbophil will be added to the aqueous phase. The two phases will be mixed in an appropriate vessel. The provided composition will be stored in a sealed container prior administering vaginally.

### Example 3: Pharmacokinetic Studies in Mice

The following examples illustrate an administering vaginally of a danazol formulation as described above in Example 2.

### Study Design

| | | Dose | | |
|---|---|---|---|---|
| | Number | of | | |
| Group | of | Active | | |
| Number | Animals | (mg) | Formulation | Route* |
| 1 | 24 (3 per | 100 | DANAZOL | INTRAVAGINAL |
| | timepoint) | | | |
| 2 | 24 (3 per | 100 | DANAZOL | ORAL |
| | timepoint) | | | |
| 3 | 24 (3 per | 600 | DANAZOL | ORAL |
| | timepoint) | | | |

*Doses administered over ~30 seconds.

### Sample Collection

| | Terminal Peritoneal Fluid and Blood Collection |
|---|---|
| Group Number | Time |
| 1-3 | 0.0333, 0.25, 0.5, 1, 2, 4, 8, and 24 hours post dose |
| | (3 animals/timepoint) |
| Anticoagulant | K₂EDTA |
| Volume/Timepoint | Maximum Obtainable |

### Study Details

Female CD-1 mice will be allowed to acclimate for a minimum of two days.

A unit dose of the danazol composition will be applied intravaginally to Group 1 mice, while an oral formulation will be delivered to mice in Groups 2 and 3 by oral gavage. All animals will be observed at dosing and each scheduled collection. All abnormalities will be recorded.

Terminal blood samples will be collected via cardiac puncture following inhalation anesthesia. Terminal peritoneal samples will be collected by paracentesis.

Sample Processing and Storage: Blood samples will be collected into a tube containing dipotassium ethylenediaminetetracetic acid (K₂EDTA) and stored on wet ice. Whole blood will be processed to plasma by centrifugation (3500 rpm at 5° C.) within 30 minutes of collection. Plasma samples will be split into 2 equal aliquots and each transferred into 96 well plates (matrix tubes) and stored at −80° C. until analysis. Administered doses will be determined gravimetrically. Peritoneal fluid will be collected using paracentesis at various time points.

Samples will be further subjected to high-performance liquid chromatography (HPLC) using a C18 column prior to MS/MS analysis. The HPLC purified samples will be contacted with human serum albumin in order to form albumin adducts, which can be detected using MS by monitoring a m/z of about 338 corresponding to the [M+H]⁻ ion of the adduct.

### Results

Pharmacokinetic parameters will be calculated using non-compartmental methods using TK/PK analysis software programs. PK analysis will be performed. All plasma and peritoneal fluid concentration data from all animals will be included in the analysis.

### Example 4: Pharmacokinetic Studies in Humans

The following example illustrates an administering vaginally of a danazol formulation as described above in Example 2 to a human subject.

| | |
|---|---|
| TYPE OF STUDY | Controlled, Comparative Study. |
| SUBJECT | Women with suspected or confirmed |
| POPULATION | Endometriosis scheduled for laparoscopy. |
| TITLE OF STUDY | A Comparative, Open-Label, Controlled, |
| | Parallel Group Study to determine |
| | Intraperitoneal fluids, tissue and serum |
| | concentrations of VML-0501 following five |
| | days of daily vaginal applications of single |
| | dose of VML-0501 (100 mg Danazol), in |
| | comparison to five days of Danazol treatment |
| | administered as an oral tablet (Danatrol) at a |
| | daily dose of 600 mg, in two groups of twelve |
| | each consisting of women with suspected or |
| | confirmed endometriosis and scheduled for |
| | laparoscopy. |
| DURATION OF | 5-7 days |
| TREATMENT PER | |
| SUBJECT | |
| OBJECTIVES | To assess the bioavailability of VML-0501 in |
| | comparison to oral Danazol in terms of: |
| | Primary study outcome: |
| | Measurement of concentrations in serum |
| | (C_{Steady State}) |
| | Measurement of concentrations in peritoneal |
| | fluid (C_{peritoneal,Steady State}) |
| | Secondary study outcome: |
| | Concentration in endometrial tissue found |
| | outside the uterus (Lesion tissue) |
| METHODOLOGY | Open-label parallel group study |
| INCLUSION | Subjects may be admitted into the study if the |
| CRITERIA | subject: |
| | Provides written (personally signed and |
| | dated) informed consent before completing |
| | any study related procedure, which can |
| | mean any assessment or evaluation that |
| | would not have formed a part of her normal |
| | medical care. |
| | Is a female who has or is suspected to have |
| | endometriosis scheduled to undergo |
| | laparoscopy. |
| | Is greater than or equal to 18 years of age |
| | and less than 42 years. |
| | Signs a specific clause on avoiding |
| | pregnancy based on the use of two |
| | effective methods for birth control |
| | following 78 days after the last |
| | dose/application. |
| | Is non-pregnant undergoing laparoscopy |
| | for confirmed or suspected endometriosis |
| | within first 10 days of her cycle. |
| | Has a body mass index (BMI) <32 kg/m2 |
| EXCLUSION | Subjects may not be admitted into the study if |
| CRITERIA | the subject: |
| | Is pregnant (positive pregnancy test) or |
| | lactating; |
| | Has evidence of drug or alcohol abuse. |
| | Has used hormonal replacement therapy or |
| | Danazol therapy within the past three |
| | months before study entry |
| | Has any of the following: Epileptic |
| | Seizure, Migraine Headache, Angina, |
| | Chronic Heart Failure, Obstruction of a |
| | Blood Vessel by a Blood Clot, Liver |
| | Problems, Kidney Disease, Pregnancy, |
| | Combined High Blood Cholesterol and |
| | Triglyceride Level, Porphyria |
| | Has undiagnosed abnormal genital |
| | bleeding |
| | Has an androgen dependent tumor |
| | Is allergic to anabolic androgenic steroid. |
| | Is a smoker |
| STUDY | Subjects are assigned to one of the two |
| MEDICATION, | treatments options and matched according to |
| DOSE AND | infertility and or proven Endometriosis. |
| ADMINISTRATION | The study medication is applied to the top of |
| | the vagina using an applicator containing 100 |
| | mg danazol for 5-7 consecutive days. |
| | Oral Danazol (Danatrol): 200 mg capsules to |
| | be taken orally thrice daily for 5-7 consecutive |
| | days. |
| CRITERIA FOR | Concentration of Danazol in the peritoneal |
| EVALUATION | fluid |
| | Concentration of Danazol in the |
| | endometrial lesion tissue |
| | Concentration of Danazol in serum. |
| | Safety in terms of adverse events and |
| | application site inspection |
| STATISTICAL | Descriptive analyses to compare |
| METHODS | concentrations of Danazol between the |
| | groups in Serum, Peritoneal fluid, and |
| | Lesion tissue. |
| | Adverse Event Analysis |

An exemplary danazol formulation as described above with respect to Example 2 is administered for three months. Then after a 30-day period, pregnancy is allowed to occur. (Since Danazol can a 9.7 hour half-life, it can be expected that 3 days may be required to have zero drug concentration in the body).

Blood samples are collected time dependently analogous to Example 3 to determine PK parameters for danazol in circulation. Peritoneal fluid samples are collect by paracentesis at various time points.

The subject samples are processed in the manner described above with respect to Example 3. The amount of danazol can be determined in the blood and peritoneal fluid samples using mass spectrometry as described above.

### Example 5: Treatment of Endometriosis

Subjects previously diagnosed with endometriosis will be administered vaginally a pharmaceutical composition comprising danazol as described above with respect to Example 2. Clinical observation of safety parameters including hemodynamic stability, acute immune response, cardiotoxicity, respiratory function, hepatotoxicity, kidney toxicity, and others will be closely monitored. Blood samples will be collected for toxicity analyses.

Treatment will be conducted for 30 days, with daily administration of the pharmaceutical composition to the subject. An amount of endometrial deposits will be monitored in order to determine the efficacy of the treatment at ameliorating the condition.

### Example 6: Ability to Become Pregnant after Treatment

Subjects previously administered vaginally a pharmaceutical composition comprising danazol in Example 5 may be further monitored after the 30 day treatment is complete. Improvement of number of mature oocytes, implantation rates and pregnancy rates following treatment with danazol administered vaginally will be assessed to confirm fertility.

### Example 7: Treatment of Ovarian Cancer

A composition comprising doxorubicin will be prepared analogous to the hormone or danazol formulations described in Examples 1 and 2. The composition will be formulated such that applying a unit dose of the composition results in an application of about 100 mg of doxorubicin to the vagina.

Subjects will receive a unit dose of the doxorubicin composition applied intravaginally. Each subject will be administered the respective treatment for 30 days. After each administration, each subject will be monitored for a reduction in ovarian tumor size using ultrasound in order to determine the efficacy of the vaginal and oral administration.

### Example 8: Monitoring of Cardiotoxicity

Subjects administered doxorubicin by either vaginal or oral administration in Example 7 will be monitored for signs of cardiotoxicity during and after the course of treatment. Blood will be collected from each subject daily of the course of the 30 day treatment. The level of cardiac troponin I and cardiac troponin T, which are biomarkers indicative of cardiac damage, will be assessed in each subject in a blood immunoassay specific for each biomarker. Samples from subject who were not previously administered doxorubicin will be included as controls. The level of cardiotoxicity for subjects administered doxorubicin vaginally or orally will be assessed by monitoring an increase in either one of both of cardiac troponin I and cardiac troponin T over the course of treatment, which will be compared to the samples from subjects not administered doxorubicin.

### Example 9: Treatment of an Pelvic Inflammatory Disease

A composition comprising the antibiotic appropriate to the microbium causing the disease can be formulated analogous to the hormone or danazol formulations described in Examples 1 and 2.

A unit dose of the composition comprising levofloxacin can applied intravaginally to a subject previously diagnosed with an intraperitoneal infection using an applicator. The composition can be formulated such that applying a unit dose of the composition results in an application of about 400 mg of the antibiotic to the vagina. After application, the subject will be monitored for amelioration of the infection.

### Example 10: Delivery of Active Ingredients to Peritoneal Cavity

The following example illustrates an administering of either a vaginal or oral formulation of danazol to female human subjects. The vaginal formulation was prepared as described above in Example 2. The oral formulation contained a total dose of 200 mg of danazol present in a capsule body of titanium dioxide and gelatin, and formulated with starch, lactose, talcum and magnesium stearate.

Subjects were administered danazol daily for five days. Six subjects were given a total dose of 600 mg of the oral danazol formulation (3×200 mg) once daily for five days. Seven subjects were given the vaginal formulation containing a total of 100 mg danazol. Samples were then taken from each subject in order to determine the concentration of danazol present systemically, in peritoneal fluid, and in peritoneal tissue. In order to quantify the amount of danazol present systemically, serum samples were collected from each subject and analyzed as described above in Example 3. In order to determine the amount of danazol present in the peritoneal fluid and tissue, endometrial lesions or suspected lesions were harvested, and the amount of danazol present in the samples was quantified as described in Example 3. Results are shown in Table 2.

TABLE 2

| | | | Vaginal - 100 mg | | |
|---|---|---|---|---|---|
| | Oral - 600 mg Danazol | | Danazol | | Estimated |
| | | Peritoneal | | Peritoneal | Peritoneal |
| | Systemic | Fluid | Systemic | Fluid | Tissue |
| | (ng/mL) | (ng/mL) | (ng/mL) | (ng/mL) | (ng/mg) |
| | 198.3 | 130.7 | 7.3 | 1 | 29.2 |
| | 113.4 | 67.7 | 4.4 | 1 | 17.6 |
| | 209 | 134 | 2.1 | 1 | 8.4 |
| | 141.3 | 16.2 | 3.3 | 1 | 13.2 |
| | 167 | 132.8 | 3.4 | 1.2 | 13.6 |
| | 109.7 | 19.2 | 4.3 | 1.6 | 17.2 |
| | — | | 4.1 | 1.8 | 16.4 |
| Average | 156.5 | 83.4 | 4.1 | 1.2 | 16.5 |
| Standard | 42.2 | 56.8 | 1.8 | 0.2 | 6.4 |
| Deviation | | | | | |
| Variance | 1778.5 | 3224.4 | 2.6 | 0.1 | 41.3 |

While embodiments have been shown and described herein, it will be obvious to those skilled in the art that such embodiments are provided by way of example only. Numerous variations, changes, and substitutions can occur to those skilled in the art.

1. A method comprising administering vaginally to a subject a pharmaceutical composition in the form of an emulsion comprising an active agent or salt thereof and a bioadhesive; wherein the pharmaceutical composition is administered at a dose of from about 10 mg to about 400 mg of the active agent or salt thereof; and wherein the administering vaginally of the pharmaceutical composition produces a substantially zero order release rate profile of the active agent into a peritoneal cavity of the subject at least about 8 hours after the administering of the pharmaceutical composition.

2. The method of claim 1, wherein the dose comprises from about 50 mg to about 100 mg of the active agent or salt thereof.

3. The method of claim 1 or 2, wherein the active agent or salt thereof is present in the peritoneal cavity at about 12 hours after the administering of the pharmaceutical composition.

4. A method comprising administering vaginally to a subject a pharmaceutical composition in the form of an emulsion comprising: an active agent or salt thereof and a bioadhesive; wherein the administering vaginally of the pharmaceutical composition comprising an administering of a dose of the active agent or salt thereof; and wherein the administering vaginally at least partially minimizes a side effect relative to an administering orally of an oral pharmaceutical composition comprising a substantially equivalent dose of the active agent or salt thereof.

5. The method of claim 4, wherein the administering vaginally is performed at least 1, 2, 4, or 6 times within 24 hours.

6. The method of claim 5, wherein the side effect is selected from the group consisting of cardiotoxicity; renal toxicity; hepatotoxicity; and any combination thereof as determined by a lower amount of a biomarker implicated in the side effect after the administering vaginally of the pharmaceutical composition relative to an amount of the biomarker implicated in the side effect after the administering orally of the oral pharmaceutical composition.

7. The method of any one of claims 1-6, wherein the administering vaginally of the pharmaceutical composition produces a peritoneal concentration of the active agent, a metabolite thereof, or a salt thereof that is at least about 4-fold greater than a peritoneal concentration of the active agent, metabolite thereof, or salt thereof achieved through an oral administering of an oral pharmaceutical composition comprising a substantially equivalent dosage of the active agent or salt thereof.

8. The method of any one of claims 1-7, which is a method of treating a disease or condition.

9. The method of claim 8, wherein the disease or condition is selected from the group consisting of an endometrial disorder, a cancer, an inflammatory disorder, an infection, and any combination thereof.

10. The method of claim 9, wherein the disease or condition is an endometrial disorder.

11. The method of claim 10, wherein the endometrial disorder is endometriosis, adenomyosis, or a combination thereof.

12. The method of claim 10 or 11, wherein an amount of an endometrial deposit is lower after the administering vaginally of the pharmaceutical composition than an amount prior to the administering vaginally of the pharmaceutical composition.

13. The method of claim 9, wherein the disease or condition is a cancer.

14. The method of claim 13, wherein the cancer is selected from the group consisting of cervical cancer; ovarian cancer; mesothelial cancer; peritoneal cancer; and any combination thereof.

15. The method of claim 13 or 14, wherein the treating comprises a reduction of a tumor size or a reduction of a tumor growth.

16. The method of claim 15, wherein the reduction of the tumor size or the reduction of the tumor growth is determined by a reduction in a tumor volume as measured by ultrasound.

17. The method of claim 9, wherein the disease or condition is an inflammatory disorder.

18. The method of claim 17, wherein the inflammatory disorder is selected from the group consisting of: pelvic inflammatory disease; chronic pelvic pain; and any combination thereof.

19. The method of claim 17 or 18, wherein the treating comprises reducing an amount of at least one pro-inflammatory cytokine to an amount that is lower than prior to the administering vaginally of the pharmaceutical composition.

20. The method of claim 9, wherein the disease or condition is an infection.

21. The method of claim 20, wherein the infection is a bacterial infection.

22. The method of claim 20, wherein the infection is a viral infection.

23. The method of claim 20, wherein the infection is a fungal infection.

24. The method of any one of claims 1-23, wherein the active agent or salt thereof is selected from the group consisting of a hormone; an antineoplastic; a GnRH agonist; a GnRH antagonist; a steroid; an antibiotic; an antiviral compound; an antifungal compound; an anti-inflammatory; a salt of any of these; and any combination thereof.

25. The method of claim 24, wherein the active agent is the hormone or a salt thereof, and wherein the hormone or salt thereof is selected from the group consisting of: testosterone; estradiol; ethinylestradiol; progesterone; levonorgestrel; oxytocin; desogestrel; a synthetic progesterone; a salt of any of these; and any combination thereof.

26. The method of claim 24, wherein the active agent is the antineoplastic or a salt thereof, and wherein the antineoplastic or salt thereof is selected from the group consisting of cyclophosphamide; methotrexate; 5-fluorouracil; doxorubicin; procarbazine; prednisolone; bleomycin; vinblastine; dacarbazine; cisplatin; epirubicin; dichloroacetate, a salt of any of these; and any combination thereof.

27. The method of claim 24, wherein the active agent is the GnRH agonist, a GnRH antagonist or a salt thereof, and wherein the GnRH agonist, GnRH antagonist, or salt thereof is selected from the group consisting of leuprolide; buserelin; histrelin; goserelin; deslorelin; nafarelin; triptorelin; cetrorelix; abarelix; ganirelix; ozarelix; degarelix; teverelix; a salt of any of these; and any combination thereof.

28. The method of claim 24, wherein the active agent is the steroid or a salt thereof, and wherein the steroid or salt thereof is danazol or a salt thereof.

29. The method of claim 24, wherein the active agent is the antibiotic or a salt thereof, and wherein the antibiotic or salt thereof is selected from the group consisting of Ceftobiprole; Ceftaroline; Clindamycin; Dalbavancin; Daptomycin; Linezolid; Mupirocin; Oritavancin; Tedizolid; Telavancin; Tigecycline; Vancomycin; an Aminoglycoside; a Carbapenem; Ceftazidime; Cefepime; Ceftobiprole; a Fluoroquinolone; Piperacillin; Ticarcillin; Linezolid; a Streptogramin; Tigecycline; Daptomycin; a salt of any of these; and any combination thereof.

30. The method of claim 24, wherein the active agent is the antiviral compound or a salt thereof, and wherein the antiviral compound or salt thereof is selected from the group consisting of Ceftobiprole; Ceftaroline; Clindamycin; Dalbavancin; Daptomycin; Linezolid; Mupirocin; Oritavancin; Tedizolid; Telavancin; Tigecycline; Vancomycin; an Aminoglycoside; a Carbapenem; Ceftazidime; Cefepime; Ceftobiprole; a Fluoroquinolone; Piperacillin; Ticarcillin; Linezolid; a Streptogramin; Tigecycline; Daptomycin; a salt of any of these; and any combination thereof.

31. The method of claim 24, wherein the active agent is the antifungal compound or a salt thereof, and wherein the antifungal compound or salt thereof is selected from the group consisting of ciclopirox olamine; haloprogin; tolnaftate; undecylenate; topical nystatin; amorolfine; butenafine; naftifine; terbinafine; a salt of any of these; and any combination thereof.

32. The method of claim 24, wherein the active agent is the anti-inflammatory or a salt thereof, and wherein the anti-inflammatory or salt thereof is selected from the group consisting of diclofenac; ketoprofen; ibuprofen; aspirin; a salt of any of these; and any combination thereof.

33. The method of any one of claims 1-32, wherein the pharmaceutical composition is administered at a dose of the active agent or salt thereof of at least at least about 50 mg, at least about 100 mg, at least about 150 mg, at least about 200 mg, at least about 250 mg, at least about 300 mg, at least about 350 mg, or at least about 400 mg.

34. The method of any one of claims 1-32, wherein the pharmaceutical composition is administered at a dose of the active agent or salt thereof per body weight of the subject of at least about 0.1 mg/kg, at least about 0.5 mg/kg, at least about 1 mg/kg, at least about 1.5 mg/kg, at least about 2 mg/kg, at least about 2.5 mg/kg, at least about 3 mg/kg, at least about 3.5 mg/kg, at least about 4 mg/kg, at least about 4.5 mg/kg, at least about 5 mg/kg, at least about 5.5 mg/kg, at least about 6 mg/kg, at least about 6.5 mg/kg, at least about 7 mg/kg, at least about 7.5 mg/kg, at least about 8 mg/kg, at least about 8.5 mg/kg, at least about 9 mg/kg, at least about 9.5 mg/kg, at least about 10 mg/kg, at least about 11 mg/kg, at least about 12 mg/kg, at least about 13 mg/kg, at least about 14 mg/kg, at least about 15 mg/kg, at least about 16 mg/kg, at least about 17 mg/kg, at least about 18 mg/kg, at least about 19 mg/kg, or at least about 20 mg/kg.

35. The method of any one of claims 1-32, wherein the pharmaceutical composition comprises a substantially uniform mixture of an organic phase and an aqueous phase.

36. The method of claim 35, wherein the organic phase comprises at least one oleogel comprising at least one oily agent and at least one water insoluble cellulose polymer.

37. The method of claim 36, wherein the water insoluble cellulose polymer is an alkyl cellulose.

38. The method of claim 37, wherein the alkyl cellulose is selected from the group consisting of methylcellulose; ethylcellulose; hydroxypropylcellulose; and any combination thereof.

39. The method of claim 36, wherein the water insoluble cellulose polymer is an alkyl carboxylic containing cellulose or a salt thereof.

40. The method of claim 39, wherein the alkyl carboxylic acid containing cellulose is a substantially non-sodium containing carboxymethylcellulose.

41. The method of claim 40, wherein the substantially non-sodium containing carboxymethylcellulose comprises from about 1% to about 10% by weight of the total weight of the pharmaceutical composition.

42. The method of claim 37, wherein the alkyl cellulose comprises from about 1% to about 10% by weight of the total weight of the pharmaceutical composition.

43. The method of claim 38 comprising the ethylcellulose, wherein the ethylcellulose comprises from about 1% to about 10% by weight of the total weight of the pharmaceutical composition.

44. The method of claim 39 comprising the alkyl carboxylic containing cellulose or salt thereof, wherein the alkyl carboxylic containing cellulose or a salt thereof comprises from about 1% to about 10% by weight of the total weight of the pharmaceutical composition.

45. The method of claim 35, wherein the aqueous phase comprises at least one aqueous gel.

46. The method of claim 45, wherein the aqueous gel further comprises at least one gelling agent.

47. The method of claim 46, wherein the at least one gelling agent is selected from the group consisting of a carbomer; a poloxamer; sodium carboxymethylcellulose; and a combination thereof.

48. The method of claim 46 or 47, wherein the at least one gelling agent comprises from about 0.1% to about 10% by weight of the total weight of the aqueous gel.

49. The method of any one of claims 46-48, wherein the at least one gelling agent comprises from about 0.01% to about 10% by weight of the total weight of the pharmaceutical composition.

50. The method of claim 36, wherein the oily agent is selected from the group consisting of: a monoglyceride; a diglyceride; a triglyceride; and any combination thereof.

51. The method of claim 36, wherein the oily agent is isolated and purified.

52. The method of claim 36, wherein the oily agent is selected from the group consisting of: a synthetic diglyceride; a synthetic triglyceride; a propylene glycol isostearate; a polyoxyethylenated oleic glyceride mixture; an oil of plant origin; and any combination thereof.

53. The method of claim 52, comprising the propylene glycol isostearate, wherein the propylene glycol isostearate comprises from about 0.2% to about 2% by weight of the total weight of the pharmaceutical composition.

54. The method of claim 52, comprising the polyoxyethylenated oleic glyceride mixture, wherein the polyoxyethylenated oleic glyceride mixture comprises from about 0.2% to about 2% by weight of the total weight of the pharmaceutical composition.

55. The method of any one of claims 35-54, wherein the organic phase is at a ratio of from about 10:90 to about 90:10 by weight with respect to the aqueous phase.

56. The method of any one of claims 1-55, wherein the bioadhesive is selected from the group consisting of: a carbomer; glyceryl monooleate; hypromellose; polycarbophil; poly(methylvinyl ether-co-maleic anhydride); a salt thereof; and a combination thereof.

57. The method of claim 56, wherein the bioadhesive is polycarbophil, a salt thereof, or a combination thereof.

58. The method of any one of claims 1-57, wherein the pharmaceutical composition comprises a concentration of an alcohol from about 0% to about 4% by weight based on the total weight of the pharmaceutical composition, and wherein the alcohol is ethanol or isopropanol.

59. The method of claim 58, wherein the concentration of alcohol is about 3.5% by weight based on the total weight of the pharmaceutical composition.

60. The method of any one of claims 1-57, wherein the active agent comprises from about 0.00001% to about 10% by weight of the total weight of the pharmaceutical composition.

61. The method of any one of claims 1-57, wherein the pharmaceutical composition comprises from about 0% to about 4% of a penetration enhancer by weight of the total weight of the pharmaceutical composition.

62. The method of any one of claims 1-57, wherein the pharmaceutical composition comprises from about 0% to about 2% of a surfactant by weight of the total weight of the pharmaceutical composition, wherein the surfactant is selected from the group consisting of non-ionic; cationic; amphoteric; zwitterionic; and any combination thereof.

63. The method of any one of claims 1-62, wherein the pharmaceutical composition is administered to the subject in unit dose form.

64. The method of any one of claims 1-62, wherein the administering vaginally of the pharmaceutical composition is performed about every hour, about every 4 hours, about every 8 hours, about every 12 hours, or about every 24 hours.

65. The method of any one of claims 1-62, wherein the administering vaginally of the pharmaceutical composition is performed about once, about twice, about 3 times, about 4 times, about 5 times, about 6 times, about 7 times, or about 8 times within 24 hours.

66. The method of any one of claims 1-62, wherein the administering vaginally of the pharmaceutical composition is performed about once, about twice, about 3 times, about 4 times, about 5 times, about 6 times, about 7 times, about 8 times, about 9 times, about 10 times, about 11 times, about 12 times, about 13 times, about 14 times, about 15 times, about 16 times, about 17 times, about 18 times, about 19 times, or about 20 times a week.

67. The method of any one of claims 1-62, wherein the administering vaginally of the pharmaceutical composition is performed about once, about twice, about 3 times, about 4 times, about 5 times, about 6 times, about 7 times, about 8 times, about 9 times, about 10 times, about 11 times, about 12 times, about 13 times, about 14 times, about 15 times, about 16 times, about 17 times, about 18 times, about 19 times, about 20 times, about 21 times, about 22 times, about 23 times, about 24 times, about 25 times, about 26 times, about 27 times, about 28 times, about 29 times, about 30 times, or about 31 times a month.

68. The method of any one of claims 1-67, wherein the pharmaceutical composition is applied with a finger.

69. The method of any one of claims 1-67, wherein the pharmaceutical composition is applied with a glove.

70. The method of any one of claims 1-67, wherein the pharmaceutical composition is applied with an applicator.

71. The method of any one of claims 1-67, wherein the administering vaginally comprises an administering intravaginally, an administering topically, an administering by suppository, or any combination thereof.

72. The method of any one of claims 1-71, wherein the pharmaceutical composition maintains a substantially stable uniform appearance over a period of about 1 year when stored in a sealed container, at about 25° C., at about 1 atm pressure, and at about 50% relative humidity.

73. A pharmaceutical composition comprising:
(a) at least one oleogel comprising at least one oily agent and at least one water insoluble cellulose polymer;
(b) at least one aqueous gel;
(c) an active agent or salt thereof; and
(d) a bioadhesive;
wherein the active agent or salt thereof is present in the pharmaceutical composition in an amount of from about 50 mg to about 400 mg.

74. The pharmaceutical composition of claim 73, wherein the at least one oleogel and the at least one aqueous gel are in the form of an emulsion.

75. The pharmaceutical composition of claim 73 or 74, wherein the active agent or salt thereof is selected from the group consisting of a hormone; an antineoplastic; a GnRH agonist; a steroid; an antibiotic; an antiviral compound; an antifungal compound; an anti-inflammatory; a salt of any of these; and any combination thereof.

76. The pharmaceutical composition of claim 75, wherein the active agent is the hormone or a salt thereof, and wherein the hormone or salt thereof is selected from the group consisting of: estradiol; ethinylestradiol; progesterone; levonorgestrel; desogestrel; a synthetic progesterone; a salt of any of these; and any combination thereof.

77. The pharmaceutical composition of claim 75, wherein the active agent is the antineoplastic or a salt thereof, and wherein the antineoplastic or salt thereof is selected from the group consisting of cyclophosphamide; methotrexate; 5-fluorouracil; doxorubicin; procarbazine; prednisolone; bleomycin; vinblastine; dacarbazine; cisplatin; epirubicin; dichloroacetate, a salt of any of these; and any combination thereof.

78. The pharmaceutical composition of claim 75, wherein the active agent is the GnRH agonist, the GnRH antagonist, or a salt thereof, and wherein the GnRH agonist, the GnRH antagonist or salt thereof is selected from the group consisting of leuprolide; buserelin; histrelin; goserelin; deslorelin; nafarelin; triptorelin; cetrorelix, abarelix; ganirelix; ozarelix; degarelix; teverelix; a salt of any of these; and any combination thereof.

79. The pharmaceutical composition of claim 75, wherein the active agent is the steroid or a salt thereof, and wherein the steroid or salt thereof is danazol or a salt thereof.

80. The pharmaceutical composition of claim 75, wherein the active agent is the antibiotic or a salt thereof, and wherein the antibiotic or salt thereof is selected from the group consisting of Ceftobiprole; Ceftaroline; Clindamycin; Dalbavancin; Daptomycin; Linezolid; Mupirocin; Oritavancin; Tedizolid; Telavancin; Tigecycline; Vancomycin; an Aminoglycoside; a Carbapenem; Ceftazidime; Cefepime; Ceftobiprole; a Fluoroquinolone; Piperacillin; Ticarcillin; Linezolid; a Streptogramin; Tigecycline; Daptomycin; a salt of any of these; and any combination thereof.

81. The pharmaceutical composition of claim 75, wherein the active agent is the antiviral compound or a salt thereof, and wherein the antiviral compound or salt thereof is selected from the group consisting of Ceftobiprole; Ceftaroline; Clindamycin; Dalbavancin; Daptomycin; Linezolid; Mupirocin; Oritavancin; Tedizolid; Telavancin; Tigecycline; Vancomycin; an Aminoglycoside; a Carbapenem; Ceftazidime; Cefepime; Ceftobiprole; a Fluoroquinolone; Piperacillin; Ticarcillin; Linezolid; a Streptogramin; Tigecycline; Daptomycin; a salt of any of these; and any combination thereof.

82. The pharmaceutical composition of claim 75, wherein the active agent is the antifungal compound or a salt thereof, and wherein the antifungal compound or salt thereof is selected from the group consisting of ciclopirox olamine; haloprogin; tolnaftate; undecylenate; topical nysatin; amorolfine; butenafine; naftifine; terbinafine; a salt of any of these; and any combination thereof.

83. The pharmaceutical composition of claim 75, wherein the active agent is the anti-inflammatory or a salt thereof, and wherein the anti-inflammatory or salt thereof is selected from the group consisting of diclofenac; ketoprofen; ibuprofen; aspirin; a salt of any of these; and any combination thereof.

84. A kit comprising a container comprising the pharmaceutical composition of any one of claims 73-83 and instructions for use.

85. A method of making a kit comprising placing the pharmaceutical composition of any one of claims 73-83 in a container.

86. A method of detecting danazol or a salt thereof in a sample comprising:
(a) contacting a portion of the sample from a subject with an albumin; and
(b) detecting danazol or a salt thereof by mass spectrometry.

87. The method of claim 86, wherein the detecting comprises a determination of an amount of an [M+H]⁺ ion comprising an m/z of 338.

88. The method of claim 87, wherein the detecting comprises a comparison of the amount of the [M+H]⁺ ion comprising the m/z of 338 to an amount of an [M+H]⁺ ion from an internal standard; wherein the internal standard is 19-Norethindrone or a salt thereof.

89. The method of claim 86, wherein the albumin is human serum albumin.

90. The method of claim 86, wherein the sample is dried and reconstituted in a buffer between (a) and (b).

91. A kit for determining an amount of danazol in a sample comprising:
(a) a sample collection vessel;
(b) an albumin; and
(c) instructions for use.

92. The kit of claim 91, further comprising an internal standard; wherein the internal standard is 19-Norethindrone or a salt thereof.

93. The kit of claim 91, further comprising a buffer.

94. The kit of claim 91, wherein the instructions for use direct a user to:
(a) collect a sample in the sample collection vessel;
(b) contact a portion of the sample with an amount of the albumin; and
(c) detect danazol or a salt thereof by mass spectrometry.
